(19) United States
(12) Patent Application Publication
(10) Pub. No.: US 2024/0325340 A1
(43) Pub. Date: Oct. 3, 2024

(54) ISOFORM SPECIFIC AGONISTS TARGETING AKT KINASE

(71) Applicants: Thomas Jefferson University, Philadelphia, PA (US); Texas Heart Institute, Houston, TX (US)

(72) Inventors: Roger S. Armen, Philadelphia, PA (US); Tung Chan, Philadelphia, PA (US); Ulrich Rodeck, Philadelphia, PA (US); Richard A. F. Dxon, Houston, TX (US); Ronald J. Biediger, Houston, TX (US)

(21) Appl. No.: 18/292,464

(22) PCT Filed: Jul. 27, 2022
(86) PCT No.: PCT/US22/38530

§ 371 Date: Jan. 26, 2024

Related U.S. Application Data
(60) Provisional application No. 63/226,283, filed on Jul. 28, 2021.

(51) Int. Cl. A61K 31/352; A61K 31/165; A61K 31/433; C07D 311/04

(52) U.S. Cl. CPC A61K 31/352; A61K 31/165; A61K 31/433; C07D 311/04

(57) ABSTRACT

The invention relates in one aspect to compounds, pharmaceutical compositions thereof, and methods using the same for selectively activating either all or a single isoform of Akt. Isoform selective-targeting is necessary for avoiding pathologies driven by concomitantly activated Akt1, Akt2 and/or Akt3.

### CROSS-REFERENCE TO RELATED APPLICATIONS

The present application claims priority under 35 U.S.C. § 119(e) to U.S. Provisional Application No. 63/226,283, filed Jul. 28, 2021, which application is hereby incorporated herein by reference in its entirety.

### BACKGROUND OF THE INVENTION

Akt (also known as protein kinase B or PKB) is a serine/threonine protein kinase with important roles in various signaling cascades. Akt acts as a major signal transducer downstream of activated phosphoinositide 3-kinase. There are three closely related isoforms of PKB in mammalian cells: Akt1 (PKBα), (Akt2) (PKBβ), and Akt3 (PKBγ). Akt is involved in regulation of diverse cellular functions such as cell proliferation, energy metabolism, and apoptosis.

The biological effects of Akt extend to multiple cell types and tissues and are, in part, isoform specific. For example, Akt1 enhances cell survival by inhibiting several apoptotic processes. Akt1 can also induce protein synthesis and is thus a key signaling protein in the cellular pathways that lead to skeletal muscle hypertrophy and general tissue growth. Akt2 is an important signaling intermediary in the insulin-signaling pathway. As opposed to the highly homologous Akt1 isoform, the Akt2 isoform specifically controls insulin-mediated glucose and lipid metabolism. Accordingly, an inherited mutation in Akt2 (R274H) is associated with severe diabetes in humans.

Due to the broad roles of Akt in tissue homeostasis and repair, there is a need for compounds and methods to selectively modulate overall Akt activity. Furthermore, due to the distinct roles of the various Akt isoforms in different tissues and disease contexts, there is a need for compounds and methods for selectively and specifically targeting a single isoform of Akt. In certain embodiments, such compounds can treat the diseases and disorders associated with that isoform and avoid pathologies driven by other Akt isoforms. The present invention addresses this unmet need.

### BRIEF SUMMARY OF THE INVENTION

In one aspect, the present disclosure relates to compounds of formula (Ia), (Ib), (II), (III), and (IV), or a salt, solvate, prodrug, enantiomer, diastereoisomer or tautomer thereof:

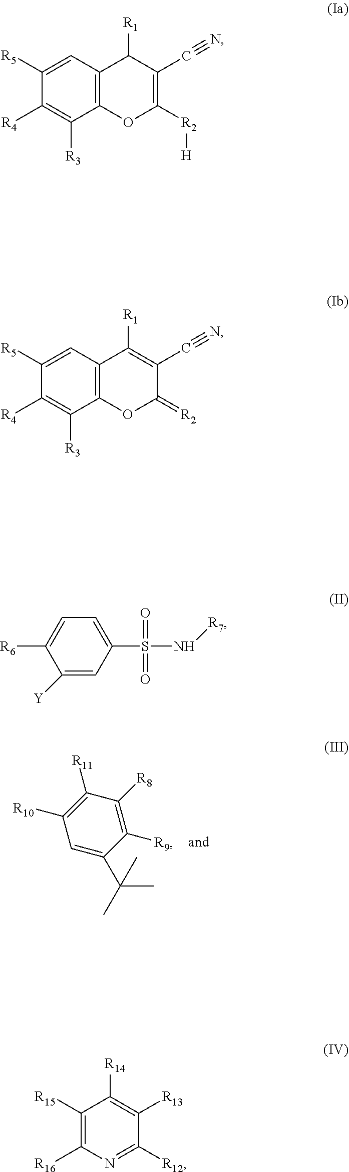

wherein R₁, R₂, R₃, R₄, R₅, R₆, R₇, R₈, R₉, R₁₀, R₁₁, R₁₂, R₁₃, R₁₄, R₁₅, and R₁₆ are defined elsewhere herein.

In another aspect, the present disclosure provides a method for promoting expansion, increasing viability, and/or altering functionality of a mammalian cell, the method comprising contacting the mammalian cell with at least one compound of formula (Ia), (Ib), (II), (III), and (IV), or a salt, solvate, prodrug, enantiomer, diastereoisomer or tautomer thereof.

In another aspect, the present disclosure provides a method of inhibiting, treating, and/or preventing symptoms or complications of a metabolic disease, pulmonary condition, diabetes, cardiovascular condition, and/or neurological/neurodegenerative disease in a subject, the method comprising administering to the subject a therapeutically effective amount of the at least one compound of formula (Ia), (Ib), (II), (III), and (IV), or a salt, solvate, prodrug, enantiomer, diastereoisomer or tautomer thereof.

In another aspect, the present disclosure provides a method of treating cancer in a subject, the method comprising:
(a) administering to the subject a therapeutically effective amount of the at least one compound of formula (Ia), (Ib), (II), (III), and (IV), or a salt, solvate, prodrug, enantiomer, diastereoisomer or tautomer thereof, or
(b) administering to the subject a therapeutically effective amount of cells pre-treated with the at least one compound of formula (Ia), (Ib), (II), (III), and (IV), or a salt, solvate, prodrug, enantiomer, diastereoisomer or tautomer thereof.

In another aspect, the present disclosure provides a method of promoting tissue replacement, promoting tissue regeneration, and/or treating degenerative diseases in a subject, the method comprising:
(b) administering to the subject a therapeutically effective amount of the at least one compound of formula (Ia), (Ib), (II), (III), and (IV), or a salt, solvate, prodrug, enantiomer, diastereoisomer or tautomer thereof, or
(b) administering to the subject a therapeutically effective amount of cells pre-treated with the at least one compound of formula (Ia), (Ib), (II), (III), and (IV), or a salt, solvate, prodrug, enantiomer, diastereoisomer or tautomer thereof.

In another aspect, the present disclosure provides a method of treating or healing a skin condition and/or a wound in a subject, the method comprising topically treating the skin and/or wound with a therapeutically effective amount of formula (Ia), (Ib), (II), (III), and (IV), or a salt, solvate, prodrug, enantiomer, diastereoisomer or tautomer thereof.

In another aspect, the present disclosure provides a method of inhibiting dephosphorylation of Akt1, Akt2 and/or Akt3, the method comprising contacting the pleckstrin homology domains of Akt1, Akt2 and/or Akt3 with the at least one compound of formula (Ia), (Ib), (II), (III), and (IV), or a salt, solvate, prodrug, enantiomer, diastereoisomer or tautomer thereof.

In another aspect, the present disclosure provides a method of (a) increasing phosphorylation states of Akt1, Akt2 and/or Akt3 in a human cell, (b) increasing catalytic activities of Akt1, Akt2 and/or Akt3 in a human cell, and/or (c) increasing phosphorylation of substrates of Akt1, Akt2 or Akt3 in a human cell, the method comprising contacting the human cell with of the at least one compound of formula (Ia), (Ib), (II), (III), and (IV), or a salt, solvate, prodrug, enantiomer, diastereoisomer or tautomer thereof.

### BRIEF DESCRIPTION OF THE DRAWINGS

The following detailed description of illustrative embodiments of the invention will be better understood when read in conjunction with the appended drawings. For the purpose of illustrating the invention, exemplary embodiments are shown in the drawings. It should be understood, however, that the invention is not limited to the precise arrangements and instrumentalities of the embodiments shown in the drawings.

FIGS. 1A-1B: illustrate the finding that 4H-chromenes SC79, HA14, and T625 block recombinant pAkt1 dephosphorylation by PP2A and cellular phosphatases. FIG. 1A: Recombinant phospho-Akt (50 ng) were pre-mixed with DMSO or with 40 μM 4H-Chromenes with carboxylate side chain (SC-79 and HA-14) or with 60 μM malononitrile side chain (T625) at 4° C. Then recombinant PP2A (70 ng PP2A-C) or total cellular phosphatase extracts were added and incubated for 30 minutes at 30° C. After incubation, protein extracts were subjected to immunoblot analysis using antibodies detecting phosphorylated Akt (T308), total Akt and PP2A catalytic subunit. FIG. 1B comprises a graph showing the percentage of Akt dephosphorylation at 30 minutes (Dephos. %) (n=6) *P<0.01 vs. DMSO.

FIGS. 2A-2B: illustrate the finding that T625's position 6 analogues differentially activate Akt and induce cellular toxicity. FIG. 2A: To distinguish heterologous-expressed Akt from endogenous Akt, a 41 amino acid large tag containing the HA-epitope was fused with wild type Akt2 (Akt2-LT WT). H9C2 myoblasts over-expressing Akt2-LT WT were treated with insulin or indicated compounds in serum-free media for 2 hr. Phosphorylation of T309 and total Akt2 are shown. FIG. 2B: Cell images show A549 AT2 cell morphology after 24 hr of drug treatment.

FIGS. 3A-3D: illustrate a model of T625 C-6 interaction space within the Akt PH domain and biochemical validation of the model. FIG. 3A: Model of the Akt2 PH-domain bound to T625 where all residue numbering corresponds to the Akt2 sequence. The C-6 Cl group act as the most optimal structural mimetic of the phosphate's electrostatic interactions with Arg23 and surrounding residues. FIG. 3B: Illustrates the finding that T625 acts as an inhibitor in E17-Y18-I19 and R₂₃-R₂₅ mutated Akt kinase, reversing the effect of T625 from activation to inhibition in the mutant. To distinguish heterologous-expressed Akt from endogenous Akt, a 41 amino acid large tag containing the HA-epitope was fused with Akt1 (Akt1-LT) and specific mutations were created by site-directed mutagenesis. H9C2 cells expressing the indicated mutant constructs were stimulated for 15 minutes with 2 μM insulin, then switch to serum-free medium with 30 μM T625 as indicated for 60 minutes. FIG. 3C: Illustrates the finding that mutations in E17 and/or 119 in Akt kinase converting the T625 response from activation to inhibition. H9C2 cells expressing the indicated mutant constructs were stimulated for 15 minutes with 2 μM insulin, then switch to serum-free medium with 20 μM T625 as indicated for 90 minutes. Phosphorylation of T308 in Akt1-LT and endogenous Akt, HA and GAPDH loading control blots are shown. FIG. 3D: Graph of specific pT308 phosphorylation in the indicated Akt1-LT constructs with or without T625 treatment. N is indicated.

FIGS. 4A-4E illustrate the conversion of Akt PH-domain small-molecule antagonists into activators. FIG. 4A: 4H-chromene activator T625 activated Akt with reduced PAINS properties compared to SC79. FIG. 4B: In a series of N-thiadiazol-arylsulfonamides, long hydrophobic alkyl chains as shown in Antagonist 19 exhibit antagonism. RA24 was identified from a series of compounds removing such large hydrophobic chains with the goal to increase activation activity. FIG. 4C: RA14 is a 3,5-di-tert-butyl-4-phenol identified from a series of compounds containing the common substructure of the antagonist SC55. FIG. 4D: RA97 is a quinoline identified from a series of pyridine-containing compounds with 2D structural similarity to T625 and the antagonist SC75. FIG. 4E: RA97 analogues T745, T748, T775, and T776 showing improved efficacy, improved length of action, and a lack of toxicity when compared to RA97.

FIG. 5 shows the screening for Akt activators predicted by chemical modifications of known PH-binding Akt antagonists using A549 epithelial cells. Using an all-atom Molecular Docking and Virtual Screening approach, over 160 small-molecules from chemical modifications of different PH-binding Akt antagonist classes were screened to identify Akt activation activity. 40 μM of each compound derived from sulfonamides, di-tert butylphenols or pyridines was incubated in human epithelial cells (A549) for 2.5 hr. Insulin was uses as positive control. Lysates were probed for phospho-T308 using ProteinSimple WES digital western system.

FIGS. 6A-6D illustrate immunoblots for Akt activators converted from non-PAINS Akt antagonists. FIG. 6A: 40 μM of each compound derived from sulfonamides, di-tert butylphenols, or pyridines were incubated in human epithelial cells (A549) for 2.5 hr. Lysates were probed for phospho-T308 and total Akt (tAkt) using ProteinSimple WES digital western system. Insulin was uses as positive control. FIG. 6B: Illustrates that RA97 analogs demonstrate improved efficacy and improved length of action in Akt activation. 40 μM of each compound was incubated in serum-starved human epithelial cells (Hacat) for 2 hr, 4 hr, or 24 hr as indicated. Cell lysates were probed for phospho-T308 and total Akt (tAkt). FIG. 6C: Illustrates that low dose T775 and T776 activate Akt and synergize with FBS. 10 μM or 20 μM of T775 or T776 were incubated with or without 0.25% fetal bovine serum (FBS) medium in human epithelial cells (Hacat) for 6 hr. Cell lysates were probed for phospho-T308, total Akt (tAkt), and GAPDH. FIG. 6D: Illustrates a T775 and T776 phosphorylated Akt substrate, PRAS40. 40 μM of each compound was incubated in serum-starved human epithelial cells (Hacat) for 6 hr. Cell lysates were probed for phospho-T308, total Akt (tAkt), Akt substrate, p-PRAS40, and GAPDH.

FIGS. 7A-7D illustrate results for Akt activator in different scaffolds in simulated ischemia and reperfusion of H9C2 cells and rat neonatal myocytes. FIG. 7A: Illustrates a schematic of simulated ischemic-reperfusion using 1% O₂ incubator, indicating drug adding time and cell harvest time. FIGS. 7B-7C: Illustrate immunoblots for cells after 2.5 hr ischemia: T625 and T620 were added to the reperfusion medium to H9C2 cells and neonatal myocytes. Insulin was used as control. FIG. 7D: Illustrates immunoblots of pT308, total Akt, GAPDH and quantified pT308/tAkt ratio for testing different Akt activator scaffolds. After ischemia, 20 μM T625 4H-chromene, 40 μM RA126 di-tert-butyl or RA97 quinoline were added to the reperfusion medium for 1.5 hrs. Lysates were probed for phospho-T308, total Akt and GAPDH using ProteinSimple WES digital western system.

FIGS. 8A-8B illustrates that 40 μM exposure of T620, T775, or T776 for 24 hours failed to show significant cellular toxicity in human A549 cells and Hacat cells. FIG. 8A: Depicts a graph illustrating that T620, which corresponds to the T625 analogue with a C-6 NO₂, reduced cellular toxicity. Cytotoxicity was determined by the release of lactate dehydrogenase (LDH) into cell culture media. A549 cells were incubated with 40 μM of each compound for 24 hours in serum-free media (n=3 for each compound). FIG. 8B: A visual assessment of cell morphology after 24 hrs treatment with 40 μM of each compound in serum-free media. 100× images are shown.

FIGS. 9A-9C illustrate the finding that T625 rescues ceramide and cathepsin G blockade of Akt activation. FIG. 9A: Illustrates the finding that C2-ceramide and cathepsin G block insulin activation of Akt. FIGS. 9B-9C: Illustrate the finding that T625 rescues ceramide and cathepsin G blockade of Akt activation by insulin in H9C2 myoblasts and in neonatal rat myocytes. H9C2 myoblasts or neonatal rat myocytes were treated with neutrophil protease Cathepsin G (0.01 U/mL), 4 hr or with 100 μM C2-Ceramide (100 μM), 2 hr. Then cells were stimulated with insulin with or without 30 μM T625 for 30 minutes. Lysates were immunoblotted with the indicated antibodies.

FIGS. 10A-10C illustrate gene expression regulated by Akt activators after ischemia and reperfusion in rat neonatal myocytes. FIG. 10A: Contains 100× cell images of neonatal myocytes after 2.5 hr ischemic-14 hr reperfusion treatment with insulin or with Akt activators (20 μM T625 or 40 μM RA126). FIGS. 10B-10C: Depicts an Affymetrix rat Clariom S transcriptome screen of rat neonatal myocytes after 2.5 hr ischemic-10 hr reperfusion treatment. Heat maps indicate genes >4× induced by insulin (FIG. 10B) or >4× repressed by insulin (FIG. 10C) and corresponding expression in Akt activator treated cells.

FIGS. 11A-11B illustrates the finding that Akt activators in human keratinocyte cell line induce VEGF secretion and enhanced cell migration. FIG. 11A: Human keratinocyte line, HaCat cells, was incubated with 20 μM of each Akt activators for 24 hours. Insulin (3.5 μg/ml) and IGF1 (100 ng/mL) treatment were used as positive control. Graph shows vascular endothelial growth factor (VEGF) secretion into culture media with indicated repeats. FIG. 11B: Akt activators enhance human keratinocyte migration. HaCaT cells were plated into silicone insert molds with a 500 μM defined cell-free gap (Ibidi) and serum-starved overnight. Then, the mold barrier was removed and cells were treated with 40 μM T620. After 22 hr, the gap spaces were imaged at 100×. Two images of control and four images of T620 treated gap spaces are shown.

FIGS. 12A-12B illustrates enhanced insulin sensitivity by T625 in vivo. 8 week old (wk) wild type mice were deprived of food overnight. Anesthetized mice were intubated on the FlexiVent system and challenged with aerosolized inhaled insulin or T625 as indicated. Lung tissue was harvested 20 minutes after drug inhalation. Extracts were immunoblotted with indicated antibodies.

FIGS. 13A-13C illustrate the finding that T620 and T625 enhanced Akt activation in mouse heart during ischemia-reperfusion procedure. FIG. 13A: A schematic of ischemic-reperfusion procedure indicating drug injection time and heart harvest time. FIG. 13B: Illustrates immunoblots of pS473, Akt phospho-substrate, pPRAS40 and GAPDH in the WES auto-western system. After ischemic-ligation, T625 (0.8 μg in 20 μl PBS) were directly injected at three spots of exposed ventricular wall. Injected insulin was used as a control. FIG. 13C: Illustrates immunoblots of pT308, total Akt (tAkt) and GAPDH and quantified pT308/tAkt ratio. After ischemic-ligation, T620 and T625 were directly injected or intraperitoneally injected.

FIGS. 14A-14B illustrate the use of an Akt activator as topical treatment for healing mouse skin wound. FIG. 14A: Shows that T620 activated Akt within mouse skin wounds. 20 μL of indicated compounds were applied within the skin wound silicone circle for 20 min. Wound tissues were harvested and blotted with anti-Akt pS473 and GAPDH using the Proteinsimple WES system. Growth factor (GF) mixture (insulin, PDGF and IGF1 growth factor) or 40 μM T620 in corn oil or in PBS as vehicles. FIG. 14B: Shows gross effects of T620 treatment on splinted murine 5 mm excisional wound. Images of WT mouse skin wound after vehicle (0.7% DMSO in PBS) or 40 μM T620 treatment at Day 3. Wound images and hematoxylin sections showing wound edge and wound bed are shown. Scale bar=400 μm.

### DETAILED DESCRIPTION OF THE INVENTION

The invention relates in one aspect to certain Akt activators, and use of such compounds, and compositions comprising same, to inhibit, treat, and/or prevent certain disorders and diseases.

### Definitions

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science, and organic chemistry are those well-known and commonly employed in the art.

As used herein, the articles “a” and “an” refer to one or to more than one (i.e. to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of 20% or +10%, more preferably +5%, even more preferably +1%, and still more preferably +0.1% from the specified value, as such variations are appropriate to perform the disclosed methods.

In one aspect, the terms “co-administered” and “co-administration” as relating to a subject refer to administering to the subject a compound of the invention or salt thereof along with a compound that may also treat any disease or disorder contemplated herein and/or with a compound that is useful in treating other medical conditions but which in themselves may cause or facilitate any disease or disorder contemplated herein. In certain embodiments, the co-administered compounds are administered separately, or in any kind of combination as part of a single therapeutic approach. The co-administered compound may be formulated in any kind of combinations as mixtures of solids and liquids under a variety of solid, gel, and liquid formulations, and as a solution.

As used herein, a “disease” is a state of health of a subject wherein the subject cannot maintain homeostasis, and wherein, if the disease is not ameliorated, then the subject's health continues to deteriorate.

As used herein, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

As used herein, the term “pharmaceutical composition” or “composition” refers to a mixture of at least one compound useful within the invention with a pharmaceutically acceptable carrier. The pharmaceutical composition can facilitate administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and bases, including inorganic acids, inorganic bases, organic acids, organic bases, solvates, hydrates, and clathrates thereof.

The term “prevent,” “preventing,” or “prevention,” as used herein, means avoiding or delaying the onset of symptoms associated with a disease or condition in a subject that has not developed such symptoms at the time the administering of an agent or compound commences. Disease, condition, and disorder are used interchangeably herein.

By the term “specifically bind” or “specifically binds,” as used herein, is meant that a first molecule preferentially binds to a second molecule (e.g., a particular receptor or enzyme), but does not necessarily bind only to that second molecule.

As used herein, a “subject” may be a human or non-human mammal or a bird. Non-human mammals include, for example, livestock and pets, such as ovine, bovine, porcine, canine, feline and murine mammals. In certain embodiments, the subject is human.

The term “treat,” “treating,” or “treatment,” as used herein, means reducing the frequency or severity with which symptoms of a disease or condition are experienced by a subject by virtue of administering an agent or compound to the subject.

By “effective amount” is meant the amount required to reduce or improve at least one symptom of a disease or disorder relative to an untreated patient. The effective amount of active compound(s) used to practice the present invention for therapeutic treatment of a disease varies depending upon the manner of administration, the age, body weight, and general health of the subject.

As used herein, the term “alkyl,” by itself or as part of another substituent means, unless otherwise stated, a straight or branched chain hydrocarbon having the number of carbon atoms designated (i.e., C₁-C₁₀ means one to ten carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, such as, but not limited to, ethyl, methyl, isopropyl, isobutyl, n-pentyl, n-hexyl and cyclopropylmethyl.

As used herein, the term “alkylene” by itself or as part of another substituent means, unless otherwise stated, a straight or branched hydrocarbon group having the number of carbon atoms designated (i.e., C₁-C₁₀ means one to ten carbon atoms) and includes straight, branched chain, or cyclic substituent groups, wherein the group has two open valencies. Examples include methylene, 1,2-ethylene, 1,1-ethylene, 1,1-propylene, 1,2-propylene and 1,3-propylene.

As used herein, the term “cycloalkyl,” by itself or as part of another substituent means, unless otherwise stated, a cyclic chain hydrocarbon having the number of carbon atoms designated (i.e., C₃-C₆ means a cyclic group comprising a ring group consisting of three to six carbon atoms) and includes straight, branched chain or cyclic substituent groups. Examples include cyclopropyl, cyclobutyl, cyclopentyl, cyclohexyl, cycloheptyl, and cyclooctyl. Most preferred is (C₃-C₆)cycloalkyl, such as, but not limited to, cyclopropyl, cyclobutyl, cyclopentyl and cyclohexyl.

As used herein, the term “alkenyl,” employed alone or in combination with other terms, means, unless otherwise stated, a stable mono-unsaturated or di-unsaturated straight chain or branched chain hydrocarbon group having the stated number of carbon atoms. Examples include vinyl, propenyl (or allyl), crotyl, isopentenyl, butadienyl, 1,3-pentadienyl, 1,4-pentadienyl, and the higher homologs and isomers. A functional group representing an alkene is exemplified by —CH₂—CH═CH₂.

As used herein, the term “alkynyl,” employed alone or in combination with other terms, means, unless otherwise stated, a stable straight chain or branched chain hydrocarbon group with a triple carbon-carbon bond, having the stated number of carbon atoms. Non-limiting examples include ethynyl and propynyl, and the higher homologs and isomers. The term “propargylic” refers to a group exemplified by —CH₂—C≡CH. The term “homopropargylic” refers to a group exemplified by —CH₂CH₂—C≡CH. The term “substituted propargylic” refers to a group exemplified by —CR₂—C≡CR, wherein each occurrence of R is independently H, alkyl, substituted alkyl, alkenyl or substituted alkenyl, with the proviso that at least one R group is not hydrogen. The term “substituted homopropargylic” refers to a group exemplified by —CR₂CR₂—C≡CR, wherein each occurrence of R is independently H, alkyl, substituted alkyl, alkenyl or substituted alkenyl, with the proviso that at least one R group is not hydrogen.

As used herein, the term “substituted alkyl,” “substituted cycloalkyl,” “substituted alkenyl” or “substituted alkynyl” means alkyl, cycloalkyl, alkenyl or alkynyl, as defined above, substituted by one, two or three substituents selected from the group consisting of halogen, C₁-C₆ alkoxy, C₁-C₆ haloalkyl, and —OH.

As used herein, the term “alkoxy” employed alone or in combination with other terms means, unless otherwise stated, an alkyl group having the designated number of carbon atoms, as defined above, connected to the rest of the molecule via an oxygen atom, such as, for example, methoxy, ethoxy, 1-propoxy, 2-propoxy (isopropoxy) and the higher homologs and isomers. Preferred are (C₁-C₃)alkoxy, such as, but not limited to, ethoxy and methoxy.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “heteroalkyl” by itself or in combination with another term means, unless otherwise stated, a stable straight or branched chain alkyl group consisting of the stated number of carbon atoms and one or more heteroatoms selected from the group consisting of O, N, and S, and wherein the nitrogen and sulfur atoms may be optionally oxidized and the nitrogen heteroatom may be optionally quaternized. The heteroatom(s) may be placed at any position of the heteroalkyl group, including between the rest of the heteroalkyl group and the fragment to which it is attached, as well as attached to the most distal carbon atom in the heteroalkyl group. Examples include: —O—CH₂—CH₂—CH₃, —CH₂—CH₂—CH₂—OH, —CH₂—CH₂—NH—CH₃, —CH₂—S—CH₂—CH₃, and —CH₂CH₂—S(═O)—CH₃. Up to two heteroatoms may be consecutive, such as, for example, —CH₂—NH—OCH₃, or —CH₂—CH₂—S—S—CH₃.

As used herein, the term “heteroalkenyl” by itself or in combination with another term means, unless otherwise stated, a stable straight or branched chain monounsaturated or di-unsaturated hydrocarbon group consisting of the stated number of carbon atoms and one or more heteroatoms selected from the group consisting of O, N, and S, and wherein the nitrogen and sulfur atoms may optionally be oxidized and the nitrogen heteroatom may optionally be quaternized. Up to two heteroatoms may be placed consecutively. Examples include —CH═CH—O—CH₃, —CH═CH—CH₂—OH, —CH₂—CH═N—OCH₃, —CH═CH—N(CH₃)—CH₃, and —CH₂—CH═CH—CH₂—SH.

As used herein, the term “aromatic” refers to a carbocycle or heterocycle with one or more polyunsaturated rings and having aromatic character, i.e. having (4n+2) delocalized π (pi) electrons, where n is an integer.

As used herein, the term “aryl,” employed alone or in combination with other terms, means, unless otherwise stated, a carbocyclic aromatic system containing one or more rings (typically one, two or three rings) wherein such rings may be attached together in a pendent manner, such as a biphenyl, or may be fused, such as naphthalene. Examples include phenyl, anthracyl, and naphthyl. Preferred are phenyl and naphthyl, most preferred is phenyl.

As used herein, the term “aryl-(C₁-C₃) alkyl” means a functional group wherein a one to three carbon alkylene chain is attached to an aryl group, e.g., —CH₂CH₂-phenyl or —CH₂-phenyl (benzyl). Preferred is aryl-CH₂— and aryl-CH(CH₃)—. The term “substituted aryl-(C₁-C₃)alkyl” means an aryl-(C₁-C₃)alkyl functional group in which the aryl group is substituted. Preferred is substituted aryl(CH₂)—. Similarly, the term “heteroaryl-(C₁-C₃)alkyl” means a functional group wherein a one to three carbon alkylene chain is attached to a heteroaryl group, e.g., —CH₂CH₂-pyridyl. Preferred is heteroaryl-(CH₂)—. The term “substituted heteroaryl-(C₁-C₃)alkyl” means a heteroaryl-(C₁-C₃)alkyl functional group in which the heteroaryl group is substituted. Preferred is substituted heteroaryl-(CH₂)—.

As used herein, the term “heterocycle” or “heterocyclyl” or “heterocyclic” by itself or as part of another substituent means, unless otherwise stated, an unsubstituted or substituted, stable, mono- or multi-cyclic heterocyclic ring system that consists of carbon atoms and at least one heteroatom selected from the group consisting of N, O, and S, and wherein the nitrogen and sulfur heteroatoms may be optionally oxidized, and the nitrogen atom may be optionally quaternized. The heterocyclic system may be attached, unless otherwise stated, at any heteroatom or carbon atom that affords a stable structure. A heterocycle may be aromatic or non-aromatic in nature. In certain embodiments, the heterocycle is an unsaturated non-aromatic heterocycle. In certain embodiments, the heterocycle is a mono-unsaturated non-aromatic heterocycle. In certain embodiments, the heterocycle is a di-unsaturated non-aromatic heterocycle. In certain embodiments, the heterocycle is a heteroaryl. Examples of heterocyclyl include thiadiazolyl, thiazolyl, oxazolyl, diazolyl, imidazolyl, triazinyl, thiazolyl, isothiazolyl, and pyridinyl.

As used herein, the term “heteroaryl” or “heteroaromatic” refers to a heterocycle having aromatic character. A polycyclic heteroaryl may include one or more rings that are partially saturated. Examples include chromene, tetrahydroisoquinoline, phenylquinoline, and tetrahydronaphthyridine

The aforementioned listing of heterocyclyl and heteroaryl moieties is intended to be representative and not limiting.

As used herein, the term “substituted” means that an atom or group of atoms has replaced hydrogen as the substituent attached to another group.

For aryl, aryl-(C₁-C₃) alkyl and heterocyclyl groups, the term “substituted” as applied to the rings of these groups refers to any level of substitution, namely mono-, di-, tri-, tetra-, or penta-substitution, where such substitution is permitted. The substituents are independently selected, and substitution may be at any chemically accessible position. In certain embodiments, the substituents vary in number between one and four. In other embodiments, the substituents vary in number between one and three. In yet other embodiments, the substituents vary in number between one and two. In yet other embodiments, the substituents are independently selected from the group consisting of C₁-C₆ alkyl, —OH, C₁-C₆ alkoxy, halo, amino, acetamido, and nitro. As used herein, where a substituent is an alkyl or alkoxy group, the carbon chain may be branched, straight or cyclic, with straight being preferred.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds and Compositions

The invention includes a compound having formula (Ia), (Ib), (II), (III), or (IV), or a salt, solvate, enantiomer, diastereoisomer, or tautomer thereof:

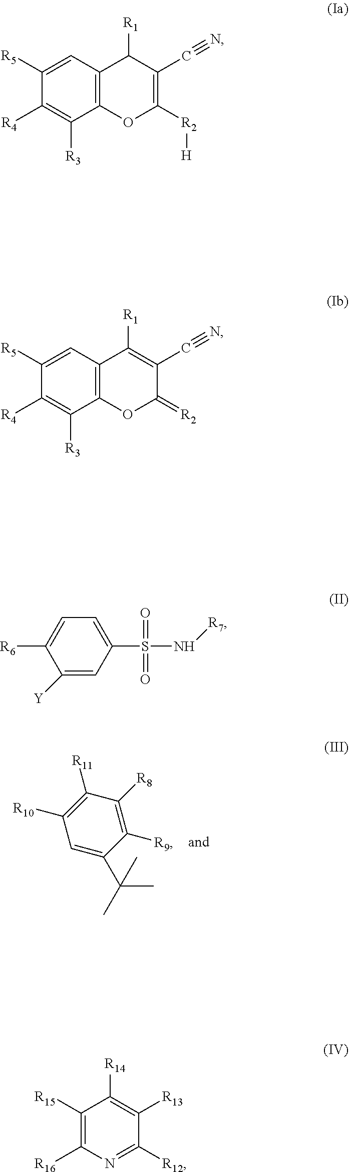

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,

wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of:

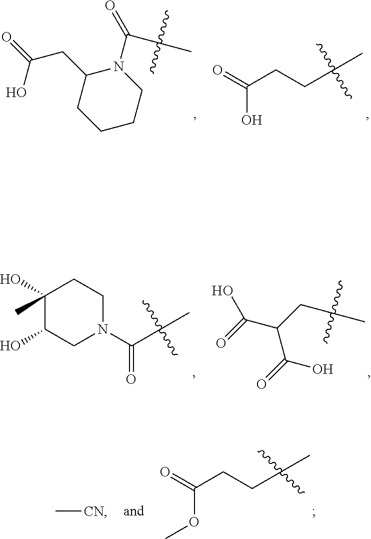

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

In certain embodiments, the compound of the invention is at least one selected from the group consisting of.

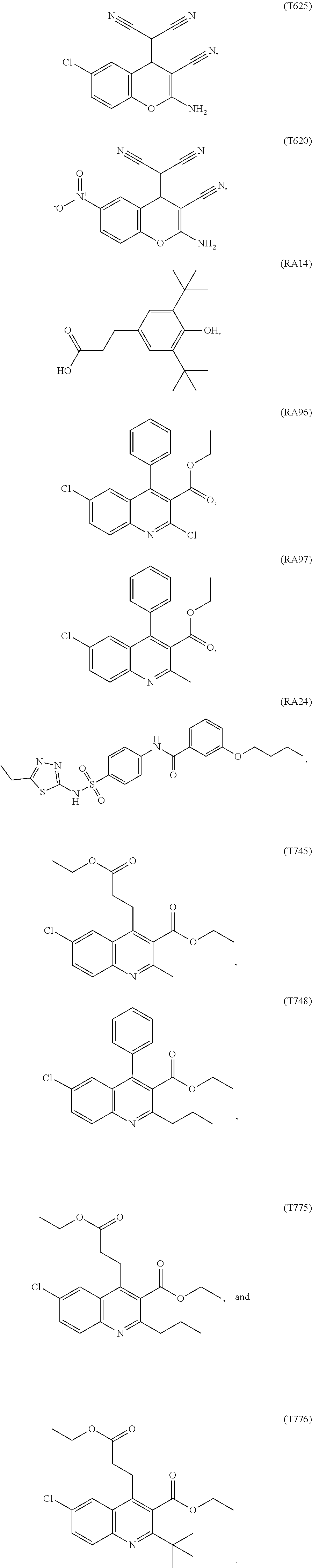

In certain embodiments, the compound of the invention is at least one selected from the group consisting of
2-(2-amino-6-chloro-3-cyano-4H-chromen-4-yl)propanedinitrile,
2-(2-amino-3-cyano-6-methoxy-4H-chromen-4-yl)propanedinitrile,
2-(2-amino-3-cyano-4H-chromen-4-yl)propanedinitrile,
2-(2-amino-6-bromo-3-cyano-4H-chromen-4-yl)propanedinitrile,
2-(2-amino-6,8-dichloro-3-cyano-4H-chromen-4-yl)propanedinitrile,
2-(2-amino-8-bromo-6-chloro-3-cyano-4H-chromen-4-yl)propanedinitrile,
2-(2-amino-6-chloro-3-cyano-8-methyl-4H-chromen-4-yl)propanedinitrile,
2-(2-amino-8-chloro-3-cyano-4H-chromen-4-yl)propanedinitrile,
2-(2-amino-7-chloro-3-cyano-4H-chromen-4-yl)propanedinitrile, 2-(2-amino-6-chloro-3-cyano-4H-chromen-4-yl)propanedinitrile,
2-(2-amino-8-bromo-3-cyano-4H-chromen-4-yl)propanedinitrile,
2-(2-amino-7-bromo-3-cyano-4H-chromen-4-yl)propanedinitrile,
2-(2-amino-3-cyano-8-methyl-4H-chromen-4-yl)propanedinitrile,
2-(2-amino-3-cyano-7-methyl-4H-chromen-4-yl)propanedinitrile,
2-(2-amino-3-cyano-6-methyl-4H-chromen-4-yl)propanedinitrile,
2-(2-amino-3-cyano-8-fluoro-4H-chromen-4-yl)propanedinitrile,
2-(2-amino-3-cyano-6-fluoro-4H-chromen-4-yl)propanedinitrile,
2-(2-amino-3-cyano-7-fluoro-4H-chromen-4-yl)propanedinitrile,
2-[2-amino-3-cyano-8-(trifluoromethyl)-4H-chromen-4-yl]propanedinitrile,
2-(2-amino-3-cyano-8-hydroxy-4H-chromen-4-yl)propanedinitrile,
2-(2-amino-3-cyano-6-nitro-4H-chromen-4-yl)propanedinitrile,
2-amino-3-cyano-4-(dicyanomethyl)-4H-chromene-8-carboxylic acid,
2-(2-amino-3-cyano-6-hydroxy-4H-chromen-4-yl)propanedinitrile,
2-(2-amino-6-chloro-3-cyano-8-hydroxy-4H-chromen-4-yl)propanedinitrile,
2-(2-amino-3-cyano-8-nitro-4H-chromen-4-yl)propanedinitrile,
2-(2-amino-6-chloro-3-cyano-4H-chromen-4-yl)propanedinitrile,
6-bromo-2-imino-2H-chromene-3-carbonitrile,
6-chloro-2-imino-2H-chromene-3-carbonitrile,
7-hydroxy-2-imino-2H-chromene-3-carbonitrile,
N-{4-[(5-ethyl-1,3,4-thiadiazol-2-yl)sulfamoyl]phenyl}acetamide,
N-{4-[(5-ethyl-1,3,4-thiadiazol-2-yl)sulfamoyl]phenyl}benzamide,
4-chloro-N-(5-ethyl-1,3,4-thiadiazol-2-yl)benzene-1-sulfonamide,
N-{4-[(5-methyl-1,3,4-thiadiazol-2-yl)sulfamoyl]phenyl}acetamide,
4-butoxy-N-{4-[(5-ethyl-1,3,4-thiadiazol-2-yl)sulfamoyl]phenyl}benzamide,
4-butyl-N-(5-methyl-1,3,4-thiadiazol-2-yl)benzene-1-sulfonamide,
3-butoxy-N-{4-[(5-ethyl-1,3,4-thiadiazol-2-yl)sulfamoyl]phenyl}benzamide,
4-chloro-N-[5-(methoxymethyl)-1,3,4-thiadiazol-2-yl]benzene-1-sulfonamide,
2-hexanoyl-N-(5-methyl-1,3,4-thiadiazol-2-yl)-1,2,3,4-tetrahydroisoquinoline-7-sulfonamide,
4-{[1,1′-biphenyl]-4-sulfonamido}benzoic acid,
N-(4-chlorophenyl)-[1,1′-biphenyl]-4-sulfonamide,
ethyl 4-{[1,1′-biphenyl]-4-sulfonamido}benzoate,
N-(5-chloropyridin-2-yl)-[1,1′-biphenyl]-4-sulfonamide,
N-(4-methyl-1,3-thiazol-2-yl)-[1,1′-biphenyl]-4-sulfonamide,
N-(5-methyl-1,2-oxazol-3-yl)-[1,1′-biphenyl]-4-sulfonamide,
N-{4-[(5-ethyl-1,3,4-thiadiazol-2-yl)sulfamoyl]phenyl}hexanamide,
3-{[1,1′-biphenyl]-4-sulfonamido}benzoic acid,
N-(pyridin-2-yl)-[1,1′-biphenyl]-4-sulfonamide,
2-[4-(3,5-di-tert-butyl-4-hydroxybenzoyl)morpholin-3-yl]acetic acid,
5-tert-butyl-2-acetamidobenzoic acid,
3-(3,5-di-tert-butyl-4-hydroxyphenyl)propanoic acid,
(3S,4R)-1-(3,5-di-tert-butyl-4-hydroxybenzoyl)-4-methylpiperidine-3,4-diol,
2-[(3,5-di-tert-butyl-4-hydroxyphenyl)methyl]propanedioic acid,
3,5-di-tert-butyl-4-hydroxybenzonitrile,
methyl 3-(3,5-di-tert-butyl-4-hydroxyphenyl)propanoate,
ethyl 2-amino-3-cyano-4-(2-methylphenyl)-5,6,7,8-tetrahydro-1,6-naphthyridine-6-carboxylate,
2-amino-6-cyclopropanecarbonyl-4-[5-(hydroxymethyl)furan-2-yl]-5,6,7,8-tetrahydro-1,6-naphthyridine-3-carbonitrile,
ethyl 2-amino-3-cyano-4-[5-(hydroxymethyl)furan-2-yl]-5,6,7,8-tetrahydroquinoline-6-carboxylate,
ethyl 2,6-dichloro-4-phenylquinoline-3-carboxylate,
ethyl 2,6-dibromo-4-phenylquinoline-3-carboxylate,
ethyl 2,6-dinitro-4-phenylquinoline-3-carboxylate,
ethyl 5-chloro-2-methyl-4-phenylquinoline-3-carboxylate,
ethyl 6-chloro-2-methyl-4-phenylquinoline-3-carboxylate,
ethyl 7-chloro-2-methyl-4-phenylquinoline-3-carboxylate,
ethyl 8-chloro-2-methyl-4-phenylquinoline-3-carboxylate,
ethyl 5-bromo-2-methyl-4-phenylquinoline-3-carboxylate,
ethyl 6-bromo-2-methyl-4-phenylquinoline-3-carboxylate,
ethyl 7-bromo-2-methyl-4-phenylquinoline-3-carboxylate,
ethyl 8-bromo-2-methyl-4-phenylquinoline-3-carboxylate,
ethyl 5-nitro-2-methyl-4-phenylquinoline-3-carboxylate,
ethyl 6-nitro-2-methyl-4-phenylquinoline-3-carboxylate,
ethyl 7-nitro-2-methyl-4-phenylquinoline-3-carboxylate,
ethyl 8-nitro-2-methyl-4-phenylquinoline-3-carboxylate,
ethyl 5-chloro-4-(morpholin-4-yl)quinoline-3-carboxylate,
ethyl 6-chloro-4-(morpholin-4-yl)quinoline-3-carboxylate,
ethyl 7-chloro-4-(morpholin-4-yl)quinoline-3-carboxylate,
ethyl 8-chloro-4-(morpholin-4-yl)quinoline-3-carboxylate,
ethyl 5-bromo-4-(morpholin-4-yl)quinoline-3-carboxylate,
ethyl 6-bromo-4-(morpholin-4-yl)quinoline-3-carboxylate,
ethyl 7-bromo-4-(morpholin-4-yl)quinoline-3-carboxylate,
ethyl 8-bromo-4-(morpholin-4-yl)quinoline-3-carboxylate,
ethyl 5-nitro-4-(morpholin-4-yl)quinoline-3-carboxylate,
ethyl 6-nitro-4-(morpholin-4-yl)quinoline-3-carboxylate,
ethyl 7-nitro-4-(morpholin-4-yl)quinoline-3-carboxylate,
ethyl 8-nitro-4-(morpholin-4-yl)quinoline-3-carboxylate,
3-(butane-1-sulfonyl)-6-chloro-4-phenylquinolin-2-ol,
2-amino-4-(5-cyano-1,2-dimethyl-1H-pyrrol-3-yl)-6-cyclopropanecarbonyl-5,6,7,8-tetrahydro-1,6-naphthyridine-3-carbonitrile,
2-amino-4-(5-chlorothiophen-2-yl)-6-cyclopropanecarbonyl-5,6,7,8-tetrahydro-1,6-naphthyridine-3-carbonitrile,
methyl 3-(2-amino-6-butanoyl-3-cyano-5,6,7,8-tetrahydro-1,6-naphthyridin-4-yl)benzoate,
ethyl 2-amino-3-cyano-4-(5-cyano-1-methyl-1H-pyrrol-3-yl)-5,6,7,8-tetrahydroquinoline-6-carboxylate,
ethyl 2-amino-3-cyano-4-(4-hydroxy-2-methoxyphenyl)-5,6,7,8-tetrahydroquinoline-6-carboxylate,
3-[5-chloro-3-(ethoxycarbonyl)-2-methylquinolin-4-yl]propanoic acid,
3-[6-chloro-3-(ethoxycarbonyl)-2-methylquinolin-4-yl]propanoic acid,
3-[7-chloro-3-(ethoxycarbonyl)-2-methylquinolin-4-yl]propanoic acid,
3-[8-chloro-3-(ethoxycarbonyl)-2-methylquinolin-4-yl]propanoic acid,
3-[5-bromo-3-(ethoxycarbonyl)-2-methylquinolin-4-yl]propanoic acid,
3-[6-bromo-3-(ethoxycarbonyl)-2-methylquinolin-4-yl]propanoic acid,
3-[7-bromo-3-(ethoxycarbonyl)-2-methylquinolin-4-yl]propanoic acid,
3-[8-bromo-3-(ethoxycarbonyl)-2-methylquinolin-4-yl]propanoic acid,
3-[5-nitro-3-(ethoxycarbonyl)-2-methylquinolin-4-yl]propanoic acid,
3-[6-nitro-3-(ethoxycarbonyl)-2-methylquinolin-4-yl]propanoic acid,
3-[7-nitro-3-(ethoxycarbonyl)-2-methylquinolin-4-yl]propanoic acid,
3-[8-nitro-3-(ethoxycarbonyl)-2-methylquinolin-4-yl]propanoic acid,
ethyl 5-chloro-4-(3-ethoxy-3-oxopropyl)-2-methylquinoline-3-carboxylate,
ethyl 6-chloro-4-(3-ethoxy-3-oxopropyl)-2-methylquinoline-3-carboxylate,
ethyl 7-chloro-4-(3-ethoxy-3-oxopropyl)-2-methylquinoline-3-carboxylate,
ethyl 8-chloro-4-(3-ethoxy-3-oxopropyl)-2-methylquinoline-3-carboxylate,
ethyl 5-bromo-4-(3-ethoxy-3-oxopropyl)-2-methylquinoline-3-carboxylate,
ethyl 6-bromo-4-(3-ethoxy-3-oxopropyl)-2-methylquinoline-3-carboxylate,
ethyl 7-bromo-4-(3-ethoxy-3-oxopropyl)-2-methylquinoline-3-carboxylate,
ethyl 8-bromo-4-(3-ethoxy-3-oxopropyl)-2-methylquinoline-3-carboxylate,
ethyl 4-(3-ethoxy-3-oxopropyl)-2-methyl-5-nitroquinoline-3-carboxylate,
ethyl 4-(3-ethoxy-3-oxopropyl)-2-methyl-6-nitroquinoline-3-carboxylate
ethyl 4-(3-ethoxy-3-oxopropyl)-2-methyl-7-nitroquinoline-3-carboxylate,
ethyl 4-(3-ethoxy-3-oxopropyl)-2-methyl-8-nitroquinoline-3-carboxylate,
ethyl 5-chloro-4-phenyl-2-propylquinoline-3-carboxylate,
ethyl 6-chloro-4-phenyl-2-propylquinoline-3-carboxylate,
ethyl 7-chloro-4-phenyl-2-propylquinoline-3-carboxylate,
ethyl 8-chloro-4-phenyl-2-propylquinoline-3-carboxylate,
ethyl 5-bromo-4-phenyl-2-propylquinoline-3-carboxylate,
ethyl 6-bromo-4-phenyl-2-propylquinoline-3-carboxylate,
ethyl 7-bromo-4-phenyl-2-propylquinoline-3-carboxylate,
ethyl 8-bromo-4-phenyl-2-propylquinoline-3-carboxylate,
ethyl 5-nitro-4-phenyl-2-propylquinoline-3-carboxylate,
ethyl 6-nitro-4-phenyl-2-propylquinoline-3-carboxylate,
ethyl 7-nitro-4-phenyl-2-propylquinoline-3-carboxylate,
ethyl 8-nitro-4-phenyl-2-propylquinoline-3-carboxylate,
3-[5-chloro-3-(ethoxycarbonyl)-2-propylquinolin-4-yl]propanoic acid,
3-[6-chloro-3-(ethoxycarbonyl)-2-propylquinolin-4-yl]propanoic acid,
3-[7-chloro-3-(ethoxycarbonyl)-2-propylquinolin-4-yl]propanoic acid,
3-[8-chloro-3-(ethoxycarbonyl)-2-propylquinolin-4-yl]propanoic acid,
3-[5-bromo-3-(ethoxycarbonyl)-2-propylquinolin-4-yl]propanoic acid,
3-[6-bromo-3-(ethoxycarbonyl)-2-propylquinolin-4-yl]propanoic acid,
3-[7-bromo-3-(ethoxycarbonyl)-2-propylquinolin-4-yl]propanoic acid,
3-[8-bromo-3-(ethoxycarbonyl)-2-propylquinolin-4-yl]propanoic acid,
3-[5-nitro-3-(ethoxycarbonyl)-2-propylquinolin-4-yl]propanoic acid,
3-[6-nitro-3-(ethoxycarbonyl)-2-propylquinolin-4-yl]propanoic acid,
3-[7-nitro-3-(ethoxycarbonyl)-2-propylquinolin-4-yl]propanoic acid,
3-[8-nitro-3-(ethoxycarbonyl)-2-propylquinolin-4-yl]propanoic acid,
ethyl 5-chloro-4-(3-ethoxy-3-oxopropyl)-2-propylquinoline-3-carboxylate,
ethyl 6-chloro-4-(3-ethoxy-3-oxopropyl)-2-propylquinoline-3-carboxylate,
ethyl 7-chloro-4-(3-ethoxy-3-oxopropyl)-2-propylquinoline-3-carboxylate,
ethyl 8-chloro-4-(3-ethoxy-3-oxopropyl)-2-propylquinoline-3-carboxylate,
ethyl 5-bromo-4-(3-ethoxy-3-oxopropyl)-2-propylquinoline-3-carboxylate,
ethyl 6-bromo-4-(3-ethoxy-3-oxopropyl)-2-propylquinoline-3-carboxylate,
ethyl 7-bromo-4-(3-ethoxy-3-oxopropyl)-2-propylquinoline-3-carboxylate,
ethyl 8-bromo-4-(3-ethoxy-3-oxopropyl)-2-propylquinoline-3-carboxylate,
ethyl 4-(3-ethoxy-3-oxopropyl)-5-nitro-2-propylquinoline-3-carboxylate,
ethyl 4-(3-ethoxy-3-oxopropyl)-6-nitro-2-propylquinoline-3-carboxylate,
ethyl 4-(3-ethoxy-3-oxopropyl)-7-nitro-2-propylquinoline-3-carboxylate,
ethyl 4-(3-ethoxy-3-oxopropyl)-8-nitro-2-propylquinoline-3-carboxylate,
3-[2-tert-butyl-5-chloro-3-(ethoxycarbonyl)quinolin-4-yl]propanoic acid,
3-[2-tert-butyl-6-chloro-3-(ethoxycarbonyl)quinolin-4-yl]propanoic acid,
3-[2-tert-butyl-7-chloro-3-(ethoxycarbonyl)quinolin-4-yl]propanoic acid,
3-[2-tert-butyl-8-chloro-3-(ethoxycarbonyl)quinolin-4-yl]propanoic acid,
3-[2-tert-butyl-5-bromo-3-(ethoxycarbonyl)quinolin-4-yl]propanoic acid,
3-[2-tert-butyl-6-bromo-3-(ethoxycarbonyl)quinolin-4-yl]propanoic acid,
3-[2-tert-butyl-7-bromo-3-(ethoxycarbonyl)quinolin-4-yl]propanoic acid,
3-[2-tert-butyl-8-bromo-3-(ethoxycarbonyl)quinolin-4-yl]propanoic acid
3-[2-tert-butyl-5-nitro-3-(ethoxycarbonyl)quinolin-4-yl]propanoic acid,
3-[2-tert-butyl-6-nitro-3-(ethoxycarbonyl)quinolin-4-yl]propanoic acid,
3-[2-tert-butyl-7-nitro-3-(ethoxycarbonyl)quinolin-4-yl]propanoic acid,
3-[2-tert-butyl-8-nitro-3-(ethoxycarbonyl)quinolin-4-yl]propanoic acid,
ethyl 2-(tert-butyl)-5-chloro-4-(3-ethoxy-3-oxopropyl)quinoline-3-carboxylate,
ethyl 2-(tert-butyl)-6-chloro-4-(3-ethoxy-3-oxopropyl)quinoline-3-carboxylate,
ethyl 2-(tert-butyl)-7-chloro-4-(3-ethoxy-3-oxopropyl)quinoline-3-carboxylate
ethyl 2-(tert-butyl)-8-chloro-4-(3-ethoxy-3-oxopropyl)quinoline-3-carboxylate,
ethyl 5-bromo-2-(tert-butyl)-4-(3-ethoxy-3-oxopropyl)quinoline-3-carboxylate,
ethyl 6-bromo-2-(tert-butyl)-4-(3-ethoxy-3-oxopropyl)quinoline-3-carboxylate,
ethyl 7-bromo-2-(tert-butyl)-4-(3-ethoxy-3-oxopropyl)quinoline-3-carboxylate,
ethyl 8-bromo-2-(tert-butyl)-4-(3-ethoxy-3-oxopropyl)quinoline-3-carboxylate,
ethyl 2-(tert-butyl)-4-(3-ethoxy-3-oxopropyl)-5-nitroquinoline-3-carboxylate,
ethyl 2-(tert-butyl)-4-(3-ethoxy-3-oxopropyl)-6-nitroquinoline-3-carboxylate,
ethyl 2-(tert-butyl)-4-(3-ethoxy-3-oxopropyl)-7-nitroquinoline-3-carboxylate, and
ethyl 2-(tert-butyl)-4-(3-ethoxy-3-oxopropyl)-8-nitroquinoline-3-carboxylate.

Selected compounds of the invention are illustrated in Table 1.

In certain embodiments, the invention includes compounds having formula (Va) or (Vb):

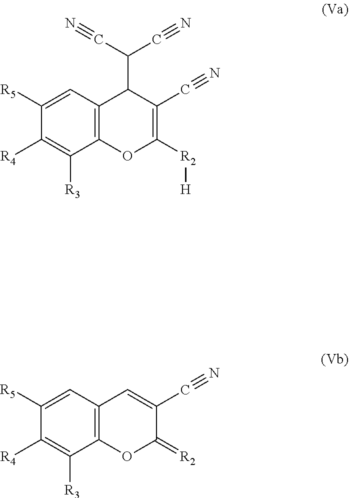

wherein:
R₂ is NH;
R₃ is selected from the group consisting of hydrogen, optionally substituted C₁-C₆ alkyl, optionally substituted C₃-C₈ cycloalkyl, optionally substituted C₂-C₆ alkenyl, optionally substituted C₂-C₆ alkynyl, hydroxyl, optionally substituted C₁-C₆ alkoxy, halogen, haloalkyl, nitro, and —C(═O)OR′, wherein R′ is selected from the group consisting of H, optionally substituted C₁-C₆ alkyl, optionally substituted C₃-C₈ cycloalkyl, optionally substituted C₂-C₈ alkenyl, and optionally substituted C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₄ is selected from the group consisting of hydrogen, halogen, optionally substituted C₁-C₆ alkyl, optionally substituted C₃-C₈ alkyl, optionally substituted C₂-C₈ alkenyl, optionally substituted C₂-C₈ alkynyl, optionally substituted C₁-C₆ alkoxy, and hydroxyl,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₅ is selected from the group consisting of hydrogen, halogen, optionally substituted C₁-C₆ alkyl, optionally substituted C₃-C₃ cycloalkyl, optionally substituted C₂-C₈ alkenyl, optionally substituted C₂-C₈ alkynyl, nitro, hydroxyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

The compounds of the invention may possess one or more stereocenters, and each stereocenter may exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography. All possible stereochemical configurations of a given compound containing chiral center(s) are contemplated. All possible mixtures enriched with a particular enantiomer or diastereomer(s) are contemplated. All pure individual enantiomers or diastereomers are contemplated.

In certain embodiments, the compounds of the invention may exist as tautomers. “Tautomerization” is a form of isomerization involving the migration of a proton accompanied by changes in bond order, often the interchange of a single bond with an adjacent double bond. Where tautomerization is possible (e.g. in solution), a chemical equilibrium of tautomers can be reached. One well known example of tautomerization is between a ketone and its corresponding enol. Heterocycles may form tautomers such as the interconversion of 2-pyrrolidinone and 3,4-dihydro-2H-5-hydroxypyrrole. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

Compounds of the invention can in certain embodiments form acids or bases. In certain embodiments, the invention contemplates acid addition salts. In other embodiments, the invention contemplates base addition salts. In yet other embodiments, the invention contemplates pharmaceutically acceptable acid addition salts. In yet other embodiments, the invention contemplates pharmaceutically acceptable base addition salts. Pharmaceutically acceptable salts refer to salts of those bases or acids that are not toxic or otherwise biologically undesirable.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds of the invention include, for example, metallic salts including alkali metal, alkaline earth metal and transition metal salts such as, for example, calcium, magnesium, potassium, sodium, lithium and copper, iron and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

The invention further include a pharmaceutical composition comprising at least one pharmaceutically acceptable carrier and at least one compound having formula (Ia), (Ib), (II), (III), or (IV), or a salt, solvate, prodrug, enantiomer, diastereoisomer or tautomer thereof.

### Methods

In one aspect, the invention provides a method for promoting expansion, increasing viability, and/or altering functionality of a mammalian cell. In certain embodiments, the method includes contacting the mammalian cell with at least one compound selected from the group consisting of formula (Ia), (Ib), (II), (III), or (IV), or a salt, solvate, prodrug, enantiomer, diastereoisomer or tautomer thereof.

Promoting cell survival and expansion is important for example, to generate large numbers of autologous immune cells in certain settings such as for CAR-T cells for immunotherapy of cancer. Similarly, adoptive T cell transfer in cancer immunotherapy requires the short-term propagation of large numbers of T cells. Additionally, recovery of hematopoiesis after bone marrow transplantation is a critical element determining the success and risks of this procedure.

In certain embodiments, the mammalian cell is an immune cell. In certain embodiments, the immune cell is an innate and/or adaptive immune cell including but not restricted to hematopoietic progenitor cell, B lymphocyte, T lymphocyte, natural killer cell, and/or myeloid lineage cell.

In certain embodiments, the mammalian cell is a keratinocyte or a vascular endothelial cell.

In certain embodiments, the at least one compound is an Akt agonist with selectivity for a single Akt isoform i.e. either Akt1, or Akt2 or Akt3. In certain embodiments, the at least one compound with selectivity for a single Akt isoform induces a unique gene expression profile upon addition to cells. In an exemplary embodiment, the at least one compound restrains access of phosphatases to phosphorylated Akt residues (T308 and S473 in Akt1) in the active Akt kinase.

In certain embodiments, the Akt isoform is activated by restraining the access of phosphatases to phosphorylated Akt residues in the active Akt kinase consequently resulting in enhanced ‘steady-state’ Akt phosphorylation and activity.

In certain embodiments, the mammalian cell is contacted ex vivo with the at least one compound. Expanding the cell ex vivo is advantageous in potentially bypassing the adverse events encountered upon administration of Akt agonists in a subject. In certain embodiments, the mammalian cell, after contacting with the at least one compound, is reintroduced in vivo. In certain embodiments, the mammalian cell contacted with the at least one compound is present in vivo.

In another aspect, the invention further provides a method of inhibiting, treating, and/or preventing symptoms or complications of a metabolic disease, pulmonary conditions, diabetes, cardiovascular conditions, and/or neurological/neurodegenerative diseases in a subject. In certain embodiments, the method comprises administering to the subject a therapeutically effective amount of the at least one compound of the invention.

In certain embodiments, the vascular conditions comprise a myocardial infarction or a stroke. In certain embodiments, the pulmonary conditions comprises an acute lung injury or a ventilator-induced lung injury. In certain embodiments, the metabolic diseases comprise acute metabolic diseases.

In yet another aspect, the invention provides a method of treating cancer in a subject. In certain embodiments, the method includes administering to the subject a therapeutically effective amount of the at least one compound of the invention or a salt, solvate, prodrug, enantiomer, diastereoisomer or tautomer thereof. In other embodiments, the method includes administering to the subject a therapeutically effective amount of cells pre-treated with the at least one compound of the invention or a salt, solvate, prodrug, enantiomer, diastereoisomer or tautomer thereof.

In yet another aspect, the invention provides a method of promoting tissue replacement, promoting tissue regeneration and/or treating degenerative diseases in a subject. In certain embodiments, the method includes administering to the subject a therapeutically effective amount of the at least one compound of the inventions or a salt, solvate, prodrug, enantiomer, diastereoisomer or tautomer thereof. In other embodiments, the method includes administering to the subject a therapeutically effective amount of cells pre-treated with the at least one compound of the inventions or a salt, solvate, prodrug, enantiomer, diastereoisomer or tautomer thereof.

The methods of the invention are thus useful for promoting expansion and/or increasing viability of an immune cell which is of therapeutic importance in, for example, cell-based immunotherapy of cancer, organ transplantation and treating immunodeficiencies.

In certain embodiments, the administration duration is equal to or less than 14 days. In certain embodiments, the administration does not cause any significant deleterious or unwanted cell multiplication in the subject.

In yet another aspect, the invention provides a method for treating or healing a skin condition and/or a wound in a subject. In certain embodiments, the method includes topically treating the skin and/or wound with a therapeutically effective amount of the at least one compound of the inventions, or a salt, solvate, prodrug, enantiomer, diastereoisomer or tautomer thereof.

In certain embodiments, the wound comprises a diabetic wound or a burn.

In certain embodiments, the treatment does not cause significant systemic exposure of the subject to at least one compound. In certain embodiments, the treatment enhances formation of new connective tissues and/or microscopic blood vessels in the subject.

In certain embodiments, the subject is a mammal. In other embodiments, the mammal is a human.

In yet another aspect, the invention provides a method of inhibiting dephosphorylation of Akt1, Akt2 and/or Akt3. In certain embodiments, the method includes contacting the pleckstrin homology domains of Akt1, Akt2 and/or Akt3 with at least one compound of the invention or a salt, solvate, prodrug, enantiomer, diastereoisomer or tautomer thereof.

In yet another aspect, the invention provides (a) increasing phosphorylation states of Akt1, Akt2 and/or Akt3 in a human cell, (b) increasing the catalytic activities of Akt1, Akt2 and/or Akt3 in a human cell, and/or (c) increasing phosphorylation of substrates of Akt1, Akt2 or Akt3 in a human cell. In certain embodiments, the method comprises contacting the human cell with the at least one compound of the invention or a salt, solvate, prodrug, enantiomer, diastereoisomer or tautomer thereof.

### Administration/Dosage/Formulations

The invention also encompasses pharmaceutical compositions and methods of their use. These pharmaceutical compositions may comprise an active ingredient (which can be one or more compounds of the invention, or pharmaceutically acceptable salts thereof) optionally in combination with one or more pharmaceutically acceptable agents. The compositions set forth herein can be used alone or in combination with additional compounds to produce additive, complementary, or synergistic effects.

The regimen of administration may affect what constitutes an effective amount. The therapeutic formulations may be administered to the subject either prior to or after the onset of a disease or disorder contemplated herein. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat a disease or disorder contemplated herein. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder contemplated herein. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect, and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated herein.

In certain embodiments, the compositions of the invention are formulated using one or more pharmaceutically acceptable excipients or carriers. In certain embodiments, the pharmaceutical compositions of the invention comprise a therapeutically effective amount of a compound of the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

In certain embodiments, the compositions of the invention are administered to the patient in dosages that range from one to five times per day or more. In other embodiments, the compositions of the invention are administered to the patient in range of dosages that include, but are not limited to, once every day, every two, days, every three days to once a week, and once every two weeks. It is readily apparent to one skilled in the art that the frequency of administration of the various combination compositions of the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physician taking all other factors about the patient into account.

Compounds of the invention for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments there between.

In certain embodiments, the dose of a compound of the invention is from about 1 mg and about 2,500 mg. In other embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in other embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present invention is directed to a packaged pharmaceutical composition comprising a container holding a therapeutically effective amount of a compound of the invention, alone or in combination with a second pharmaceutical agent; and instructions for using the compound to treat, prevent, or reduce one or more symptoms of a disease or disorder contemplated herein.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In certain embodiments, the compounds of the invention are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound of the present invention depends on the age, sex and weight of the patient, the current medical condition of the patient and the progression of a disease or disorder contemplated herein in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the inhibitor of the invention is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the viral load, to a level at which the improved disease is retained. In certain embodiments, patients require intermittent treatment on a long-term basis upon any recurrence of symptoms and/or infection.

The compounds for use in the method of the invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

Based on their structures the compound classes are divided into two broad categories Pan-Assay Interference Compounds (PAINS) and non-PAINS and four different scaffolds.

### Example 1: 4H-Chromenes and Derivatives Thereof

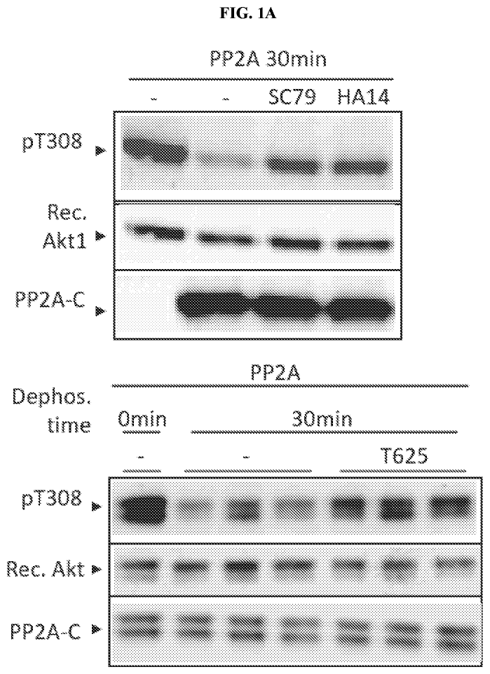

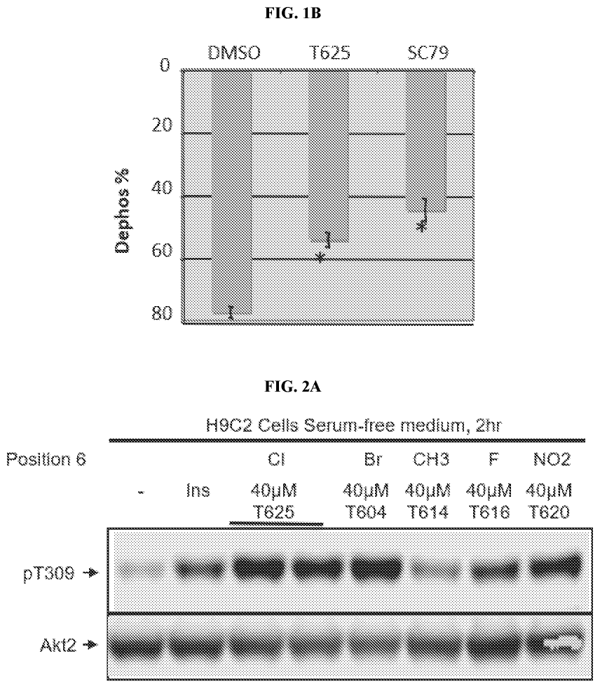

SC79 and HA14 are 4H-chromene small molecules that bind to the Akt PH-domain and activate Akt kinase in cells. It was discovered that SC79 and HA14 enhance intrinsic resistance of the Akt kinase to dephosphorylation (FIGS. 1A-1B). Without wishing to be bound by theory, it has been postulated that this is the mechanism by which 4H-chromenes enhance steady-state Akt phosphorylation and activity in cells. However, as expected of 4H-chromene compounds on the Pan-Assay Interference Compounds (PAINS) list, it was found that both SC79 and HA14 were highly labile and toxic to cells.

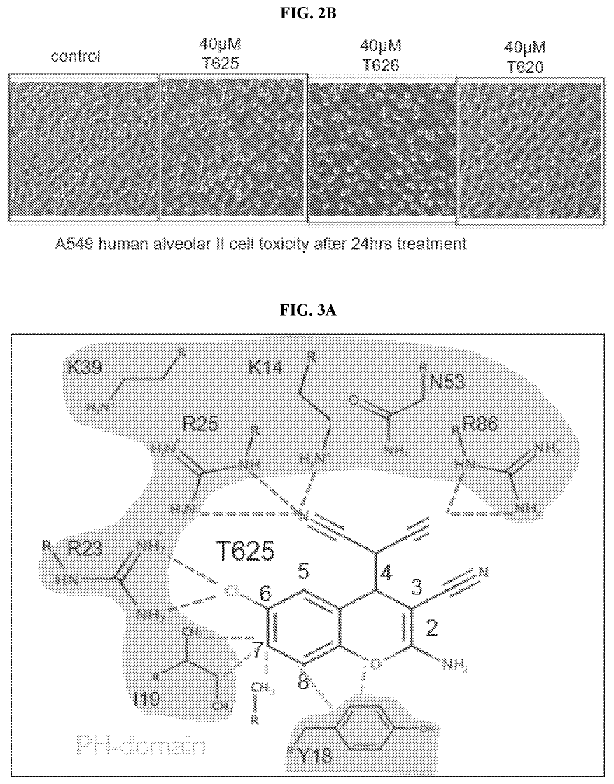

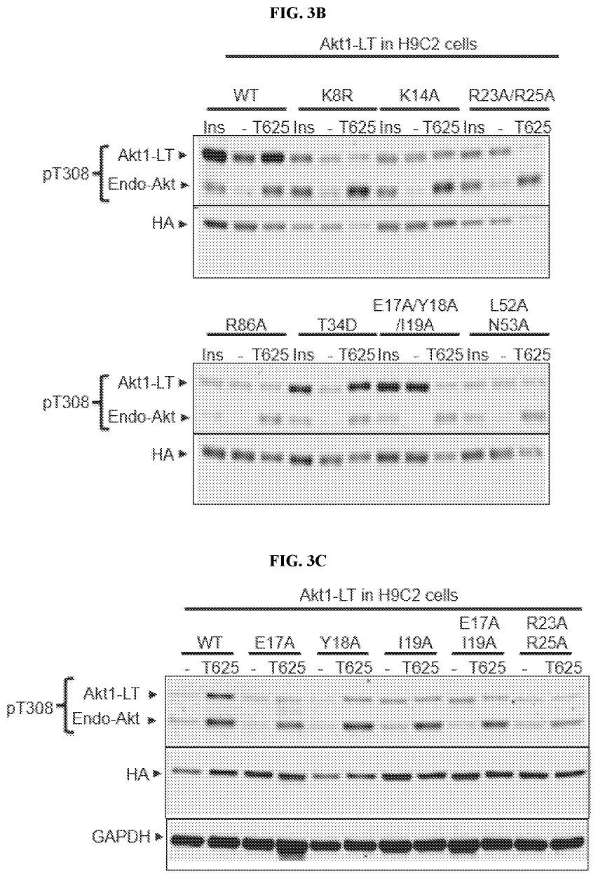

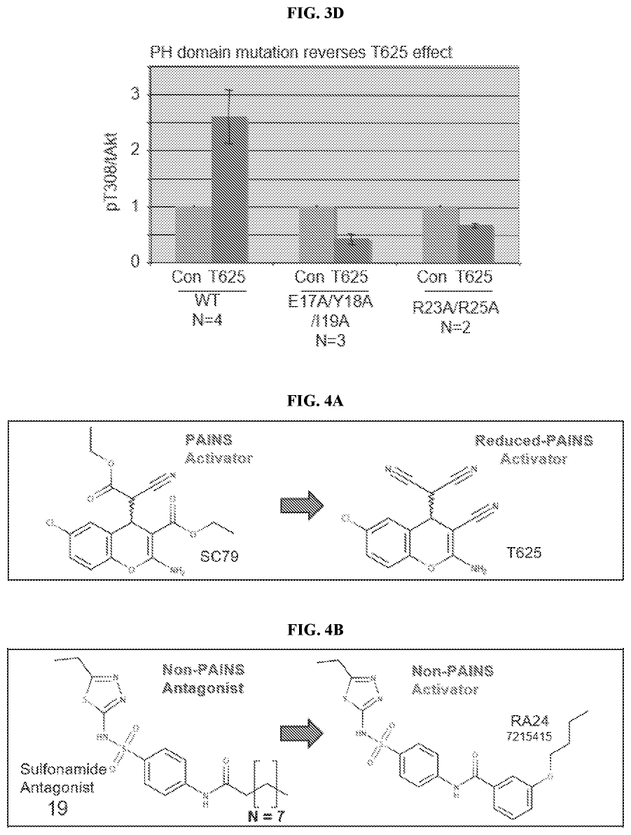

To determine whether Akt activation could be separated from cellular toxicity, a series of (>20) 4H-chromenes with nitrile, ethyl acetate and carboxylate replacements were tested. It was found that replacing the carboxylate side chain in SC79 and HA14 with malononitrile to form 2-(2-amino-6-chloro-3-cyano-4H-chromen-4-yl)propanedinitrile (T625) dramatically reduced cellular toxicity and enhanced compound stability, while retaining Akt activation in cells (FIG. 2A) and in mice (FIG. 12A). Model of T625 interaction space within the Akt PH domain suggests the C-6 Cl group acts as the most optimal structural mimetic of the phosphate's electrostatic interactions with Arg23 and surrounding residues (FIG. 3A). The finding that mutations in E17 and/or 119 in Akt kinase converting the T625 response from activation to inhibition provides biochemical validation of the model (FIGS. 3B-3D).

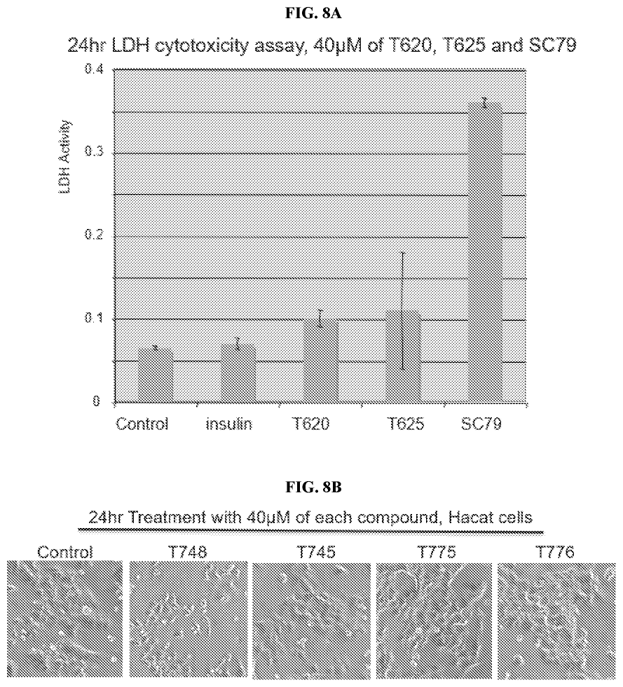

This work established that replacing the C-6 position Cl with NO₂ to form 2-(2-amino-3-cyano-6-nitro-4H-chromen-4-yl)propanedinitrile preserved Akt activation (FIG. 2A) and greatly reduced toxicity (FIG. 2B). It was confirmed that 2-(2-amino-3-cyano-6-nitro-4H-chromen-4-yl)propanedinitrile (T620) possesses low toxicity using LDH toxicity assay (FIGS. 8A-8B). Thus, both (T625) and T620 activate Akt with reduced toxicity.

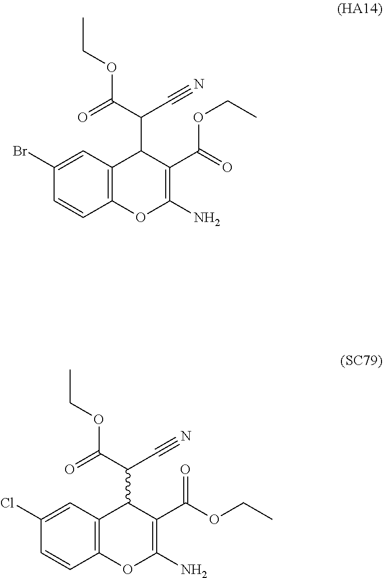 [SMILES: CCOC(=O)C1=C(N)Oc2ccc(Br)cc2C1C(C#N)C(=O)OCC ; CCOC(=O)C1=C(N)Oc2ccc(Cl)cc2C1C(C#N)C(=O)OCC]

### Example 2: 4H-Chromenes and Derivatives Thereof

A structure-based approach to identify Akt activation activity in non-PAINS classes that mimic the activity of T625 was used. Initially, this approach was guided by the prior observation that non-PAINS scaffold Akt inhibitors classes bind to the Akt PH domain. With the knowledge that Cl or nitro (NO₂) groups at the C-6 position would contribute to Akt activator activity, numerous compound classes were considered as candidates to develop activators, additional Akt activators were predicted by chemical modifications of known PH-binding Akt antagonists. Using an all-atom Molecular Docking and Virtual Screening approach, acquired over 160 small-molecules were identified and acquired from diverse classes to identify Akt activation activity in non-PAIN listed scaffolds (Jo, H. et al., Proc Natl Acad Sci USA 109, 10581-6 (2012); Meuillet, E. J. et al. Mol Cancer Ther 9, 706-17 (2010)).

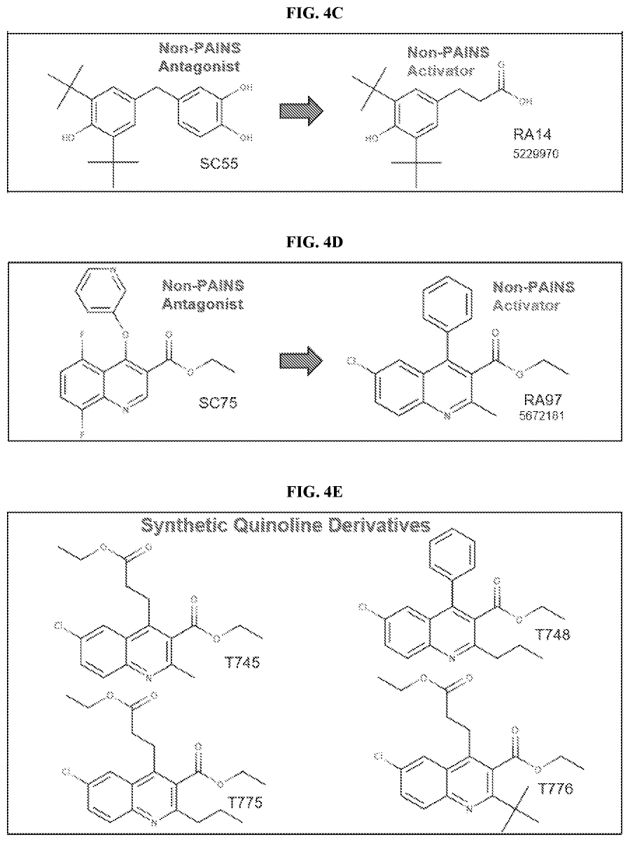

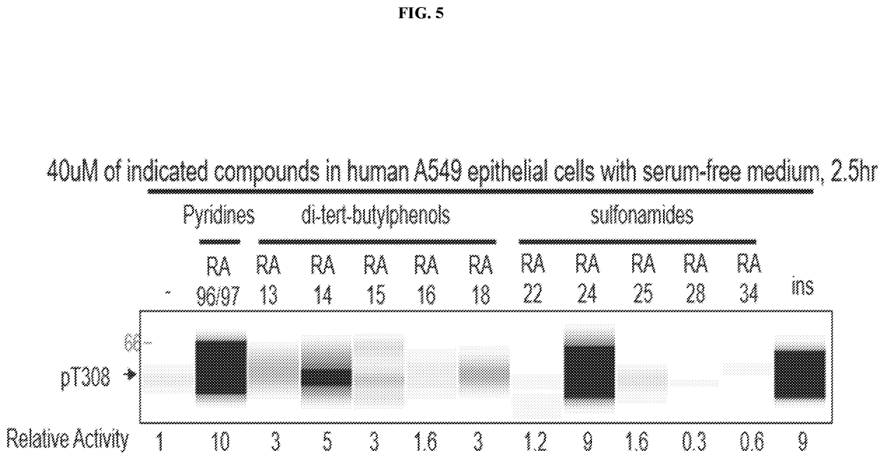

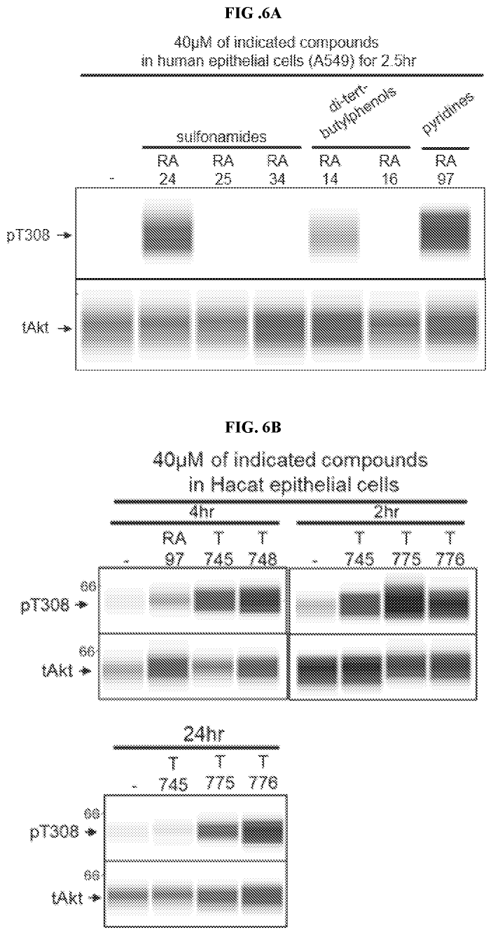

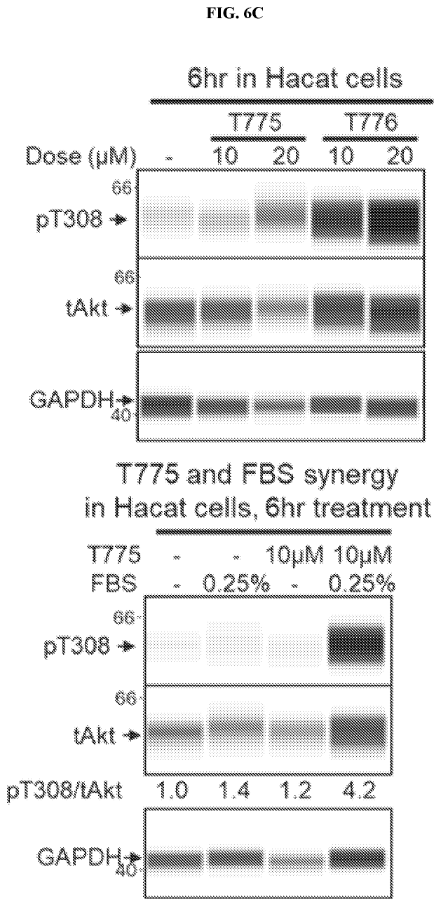

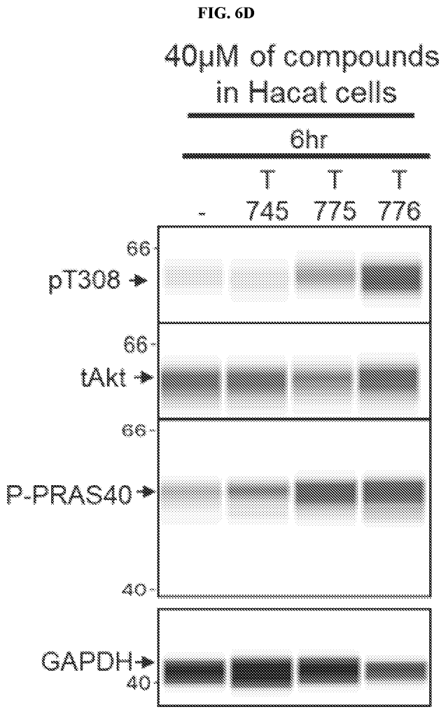

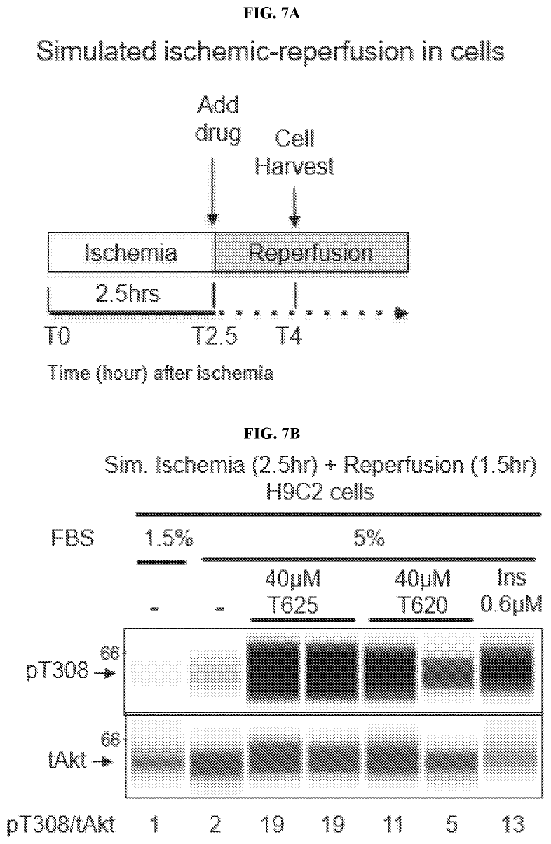

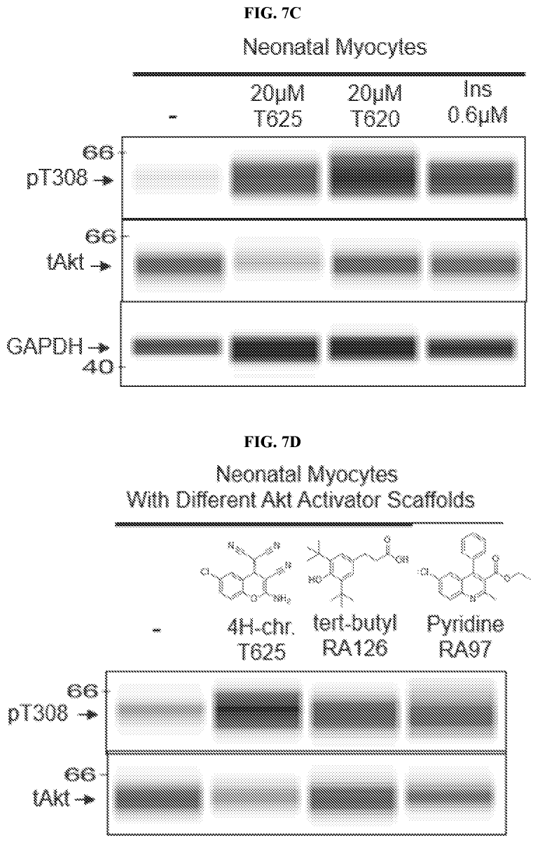

The compounds were screened for binding to Akt2 by a Surface Plasmon Resonance (SPR) direct binding assay. It was shown that at least one member of each scaffold class (sulfonamides, the di-tert-butylphenols and pyridines/quinolones; schematically shown in FIGS. 4A-4E) activated Akt kinase in human cells (FIG. 5 and FIGS. 6A-6D) and in murine cells (FIGS. 7A-7D). These compounds were biologically active as they activated Akt kinase in primary cardiomyocytes in a simulated ischemia/reperfusion injury model. This approach revealed for the first time that chemical derivatization strategy disclosed herein can be used to convert non-PAINS Akt inhibitor scaffolds into Akt activators; this is done by identifying key protein-ligand interaction contact points that strongly affect phosphatase-resistance.

Furthermore, these scaffold are more favorable than the 4H-chromenes series in reference to favorable drug-like chemistry. The non-PAINS scaffolds consist of sulfonamides, the di-tert-butylphenols and pyridines/quinolones. None of these compounds are classified within the PAINS category, and several members of these scaffolds have already been tested in animals and in humans by others.

As a near isosteric replacement that retains activity, quinolines like RA97 and analogs (T745, T748, T775, and T776) are certainly more “drug-like” than the previous 4H-chromenes, where many FDA approved drugs (e.g., montelukast, chloroquine, amodiaquine, quinidine, primaquine, and quinacrine) contain a quinoline substructure. The di-tert-butylphenols are also an established “drug-like” structural class containing previous FDA approved and experimental drugs (Probucol, SR12813, AGI-1067). These drugs are also known for exhibiting beneficial anti-oxidant properties in addition to target-specific antihyperlipidemic activity (Stocker, R. Curr Opin Lipidol 20, 227-35 (2009)). See also Flynn, D. L. et al., J Med Chem 34, 518-25 (1991).

Probucol and Succinobucol have been shown to improve insulin sensitivity and have an established drug safety record in numerous clinical trials of specific diabetic patient populations (Stocker, R. Curr Opin Lipidol 20, 227-35 (2009); Endo, K. et al., J Atheroscler Thromb 20, 494-502 (2013); Harada, Y. et al. Cardiovasc Diabetol 15, 124 (2016); Ma, X. W. et al. J Geriatr Cardiol 9, 228-36 (2012); Zhu, H. et al. J Diabetes 8, 677-85 (2016)).

The N-thiadiazol-arylsulfonamides are orally bioavailable and non-toxic in mouse models testing antagonist activity in pancreatic cancer cell xenografts in immune-compromised mice (Meuillet, E. J. et al., Mol Cancer Ther 9, 706-17 (2010); Moses, S. A. et al. Cancer Res 69, 5073-81 (2009)). Thus, compared to previous 4H-chromenes, each of the new non-PAINS scaffold classes have more attractive drug-like chemistry and established track records for oral bioavailability and minimal toxicity.

### Example 3: Testing Akt Activators in Cell Culture Models of Diseases

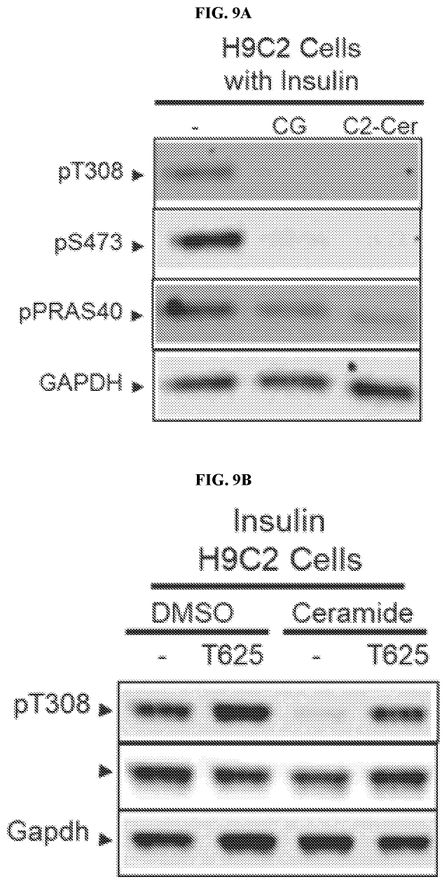

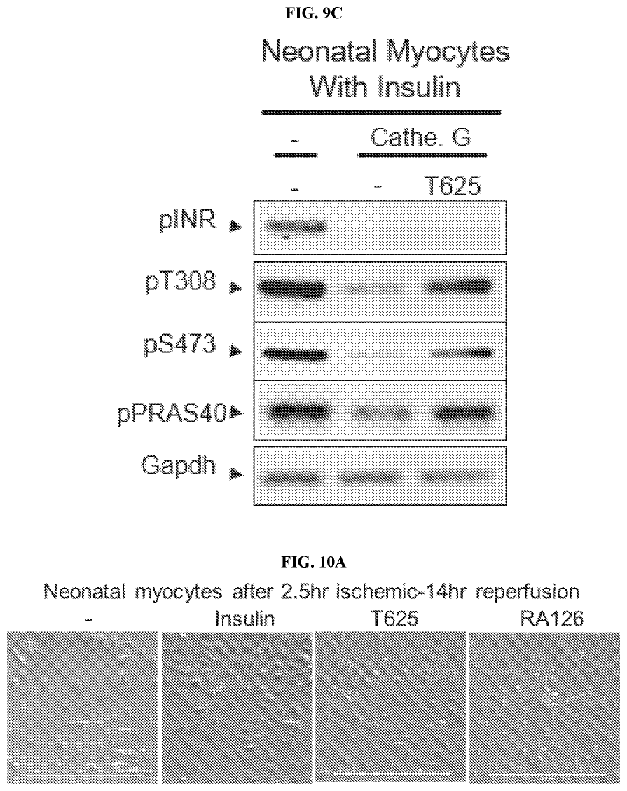

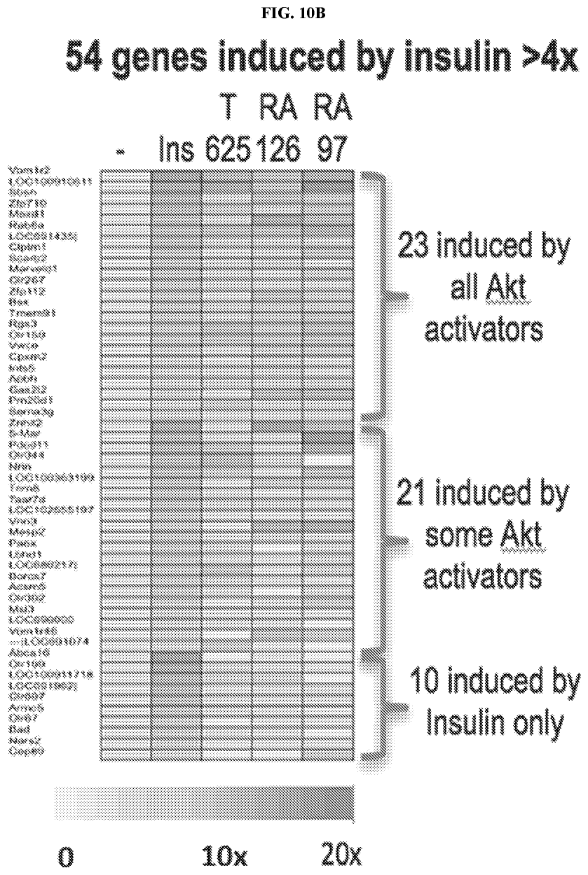

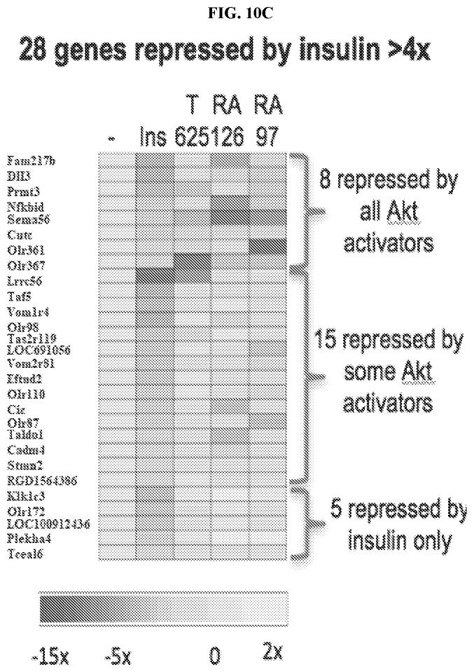

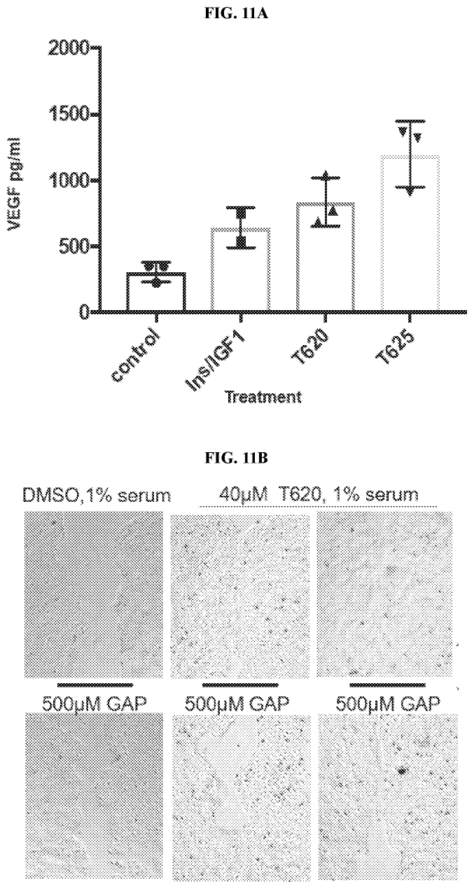

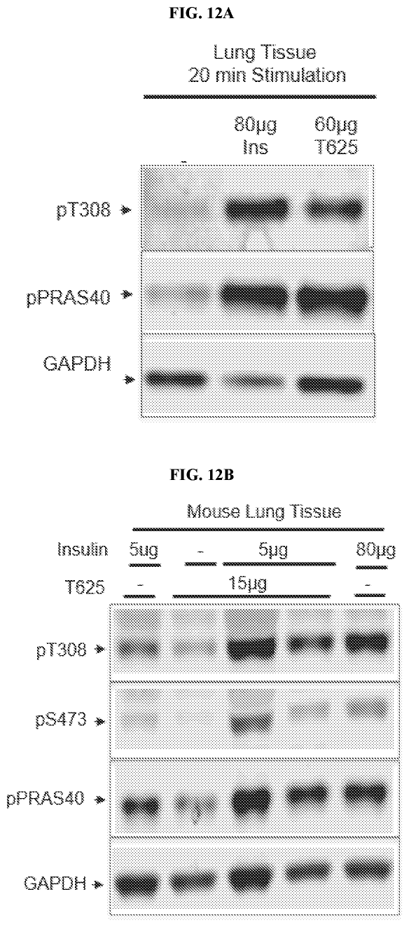

It was demonstrated that the Akt targeting compounds robustly activated Akt kinase in many human and rodent cell lines and in primary rodent cardiac myocytes, lung alveolar II epithelial cells and in human airway smooth muscle cells. Akt activators rescued Akt signaling in several cell culture model of diseases. Akt activators rescued ceramide or neutrophil derived protease cathepsin G-mediated Akt inhibition in cardiac myocytes (FIGS. 9A-9C). Akt activators enhanced Akt signaling in a simulated ischemia/reperfusion injury cell culture model (FIGS. 7A-7D). Gene expression profiling showed that 80% of insulin regulated genes were similarly regulated by Akt activator treatment (FIGS. 10A-10C). Treatment of human keratinocytes (skin epithelial cells/HaCaT) with Akt activators induced robust secretion of vascular endothelial growth factor (VEGF), a potent stimulus for angiogenesis and promoter of the epidermal keratinocyte growth during wound healing and enhanced human keratinocyte migration (FIGS. 11A-11B).

### Example 4: Testing Akt Activators in Animal Models of Disease

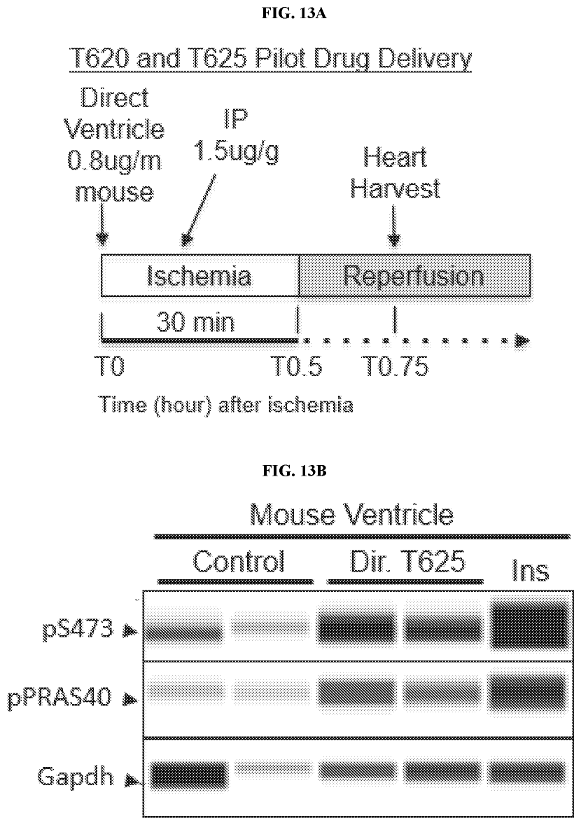

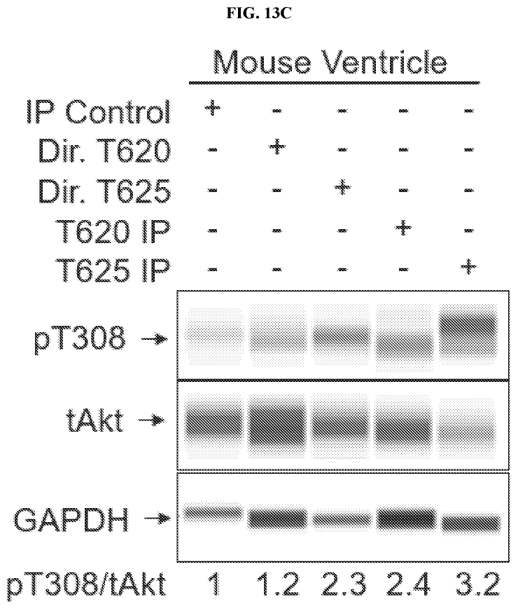

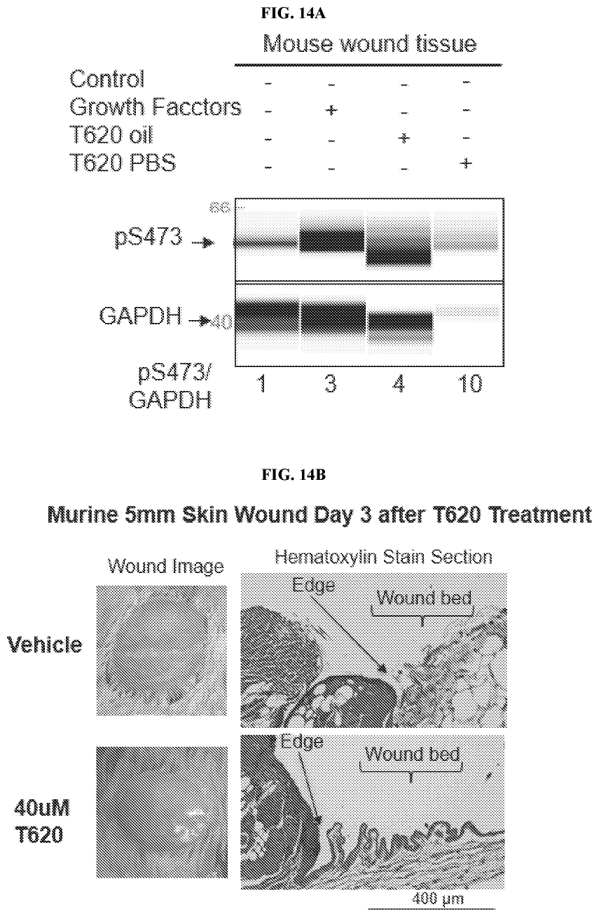

The aforementioned Akt agonists have demonstrated activity in three mouse assay models. A FlexiVent system (SCIREQ) was used to deliver Akt activator, T625, directly to mouse lung. This procedure closely mimics the clinical application of inhaled insulin. The data showed that 60 μg of 2-(2-amino-6-chloro-3-cyano-4H-chromen-4-yl)propanedinitrile robustly activated Akt kinase in mouse lung (FIGS. 12A-12B). Moreover, a suboptimal dose of (T625) (15 μg) cooperated with suboptimal insulin dose (5 μg) to activate Akt in mouse lung tissue. Akt activators were also administered in the setting of mouse cardiac ischemia-reperfusion injury model (FIGS. 13A-13C). T625 was directly injected into the left ventricular free wall at the time of ischemic ligation of exteriorized heart. Also, to mimic clinical use, (IP) Akt activators were intraperitoneally injected within 10 min after ischemia initiation. It was shown that both directly applied and intraperitoneally injected Akt activators activated Akt in the heart. Finally, a splinted murine excisional wound model that mimic human wound healing was used. Akt activators were placed directly into the wound ring, mimicking the clinical topical application of growth factors. It was shown that Akt activators enhanced Akt phosphorylation within the wound tissue (FIG. 14A), and application of Akt activator over 3 days showed a rich red granulation tissue bed within the wound (FIG. 14B), suggesting enhanced formation of new connective tissue and microscopic blood vessels.

In summary, Akt activators activated Akt in mice via inhalation, direct injection to the heart free wall and intraperitoneal systematic administration upon surgically induced heart attack and topical treatment to open wounds.

TABLE 1
Selected compounds of the present disclosure

| | |
|---|---|
| FM94 T625 | 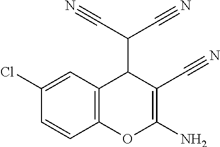 [SMILES: N#CC1=C(N)Oc2ccc(Cl)cc2C1C(C#N)C#N] |
| | 2-(2-amino-6-chloro-3-cyano-4H- |
| | chromen-4-yl)propanedinitrile |
| FM97 | 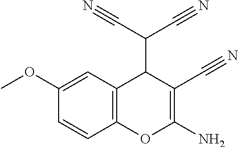 [SMILES: COc1ccc2c(c1)C(C(C#N)C#N)C(C#N)=C(N)O2] |
| | 2-(2-amino-3-cyano-6-methoxy-4H- |
| | chromen-4-yl)propanedinitrile |
| FM190 | 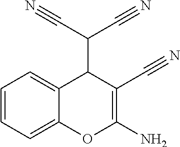 [SMILES: N#CC1=C(N)Oc2ccccc2C1C(C#N)C#N] |
| | 2-(2-amino-3-cyano-4H- |
| | chromen-4-yl)propanedinitrile |
| T604 | 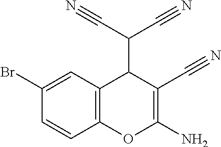 [SMILES: N#CC1=C(N)Oc2ccc(Br)cc2C1C(C#N)C#N] |
| | 2-(2-amino-6-bromo-3-cyano-4H- |
| | chromen-4-yl)propanedinitrile |
| T605 | 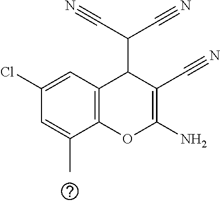 [SMILES: Cc1cc(Cl)cc2c1OC(N)=C(C#N)C2C(C#N)C#N] |
| | 2-(2-amino-6,8-dichloro-3-cyano- |
| | 4H-chromen-4-yl)propanedinitrile |
| T606 | 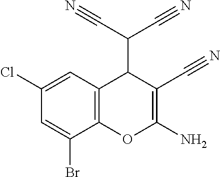 [SMILES: N#CC1=C(N)Oc2c(Br)cc(Cl)cc2C1C(C#N)C#N] |
| | 2-(2-amino-8-bromo-6-chloro- |
| | 3-cyano-4H-chromen-4-yl)- |
| | propanedinitrile |
| T607 | 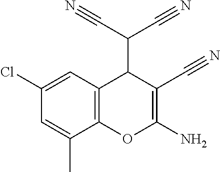 [SMILES: Cc1cc(Cl)cc2c1OC(N)=C(C#N)C2C(C#N)C#N] |
| | 2-(2-amino-6-chloro-3-cyano-8- |
| | methyl-4H-chromen-4-yl)- |
| | propanedinitrile |
| T608 | 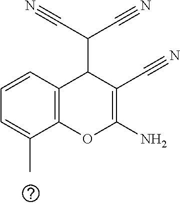 [SMILES: Cc1cccc2c1OC(N)=C(C#N)C2C(C#N)C#N] |
| | 2-(2-amino-8-chloro-3-cyano- |
| | 4H-chromen-4-yl)- |
| | propanedinitrile |
| T610 | 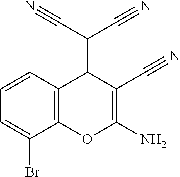 [SMILES: N#CC1=C(N)Oc2c(Br)cccc2C1C(C#N)C#N] |
| | 2-(2-amino-8-bromo-3- |
| | cyano-4H-chromen-4- |
| | yl)propanedinitrile |
| T612 | 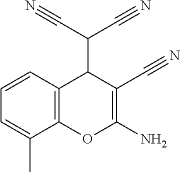 [SMILES: Cc1cccc2c1OC(N)=C(C#N)C2C(C#N)C#N] |
| | 2-(2-amino-3-cyano-8-methyl- |
| | 4H-chromen-4-yl)- |
| | propanedinitrile |
| T614 | 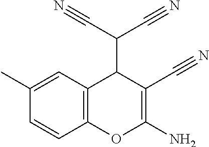 [SMILES: Cc1ccc2c(c1)C(C(C#N)C#N)C(C#N)=C(N)O2] |
| | 2-(2-amino-3-cyano-6-methyl-4H- |
| | chromen-4-yl)propanedinitrile |
| T616 | 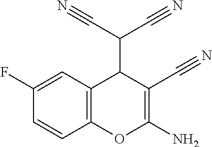 [SMILES: N#CC1=C(N)Oc2ccc(F)cc2C1C(C#N)C#N] |
| | 2-(2-amino-3-cyano-6-fluoro-4H- |
| | chromen-4-yl)propanedinitrile |
| T620 | 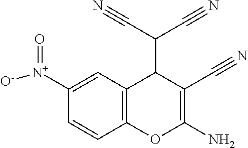 [SMILES: N#CC1=C(N)Oc2ccc([N+](=O)[O-])cc2C1C(C#N)C#N] |
| | 2-(2-amino-3-cyano-6-nitro-4H-chromen- |
| | 4-yl)propanedinitrile |
| T622 | 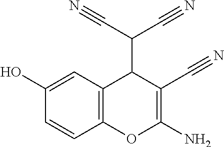 [SMILES: N#CC1=C(N)Oc2ccc(O)cc2C1C(C#N)C#N] |
| | 2-(2-amino-3-cyano-6-hydroxy-4H- |
| | chromen-4-yl)propanedinitrile |
| T625 | 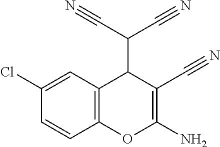 [SMILES: N#CC1=C(N)Oc2ccc(Cl)cc2C1C(C#N)C#N] |
| | 2-(2-amino-6-chloro-3-cyano-4H- |
| | chromen-4-yl)propanedinitrile |
| T603 | 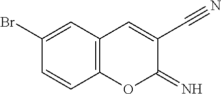 [SMILES: N#Cc1cc2cc(Br)ccc2oc1=N] |
| | 6-bromo-2-imino-2H-chromene- |
| | 3-carbonitrile |
| T626 | 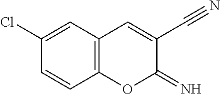 [SMILES: N#Cc1cc2cc(Cl)ccc2oc1=N] |
| | 6-chloro-2-imino-2H-chromene- |
| | 3-carbonitrile |
| T627 | 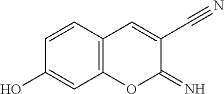 [SMILES: N#Cc1cc2ccc(O)cc2oc1=N] |
| | 7-hydroxy-2-imino-2H-chromene- |
| | 3-carbonitrile |
| RA18 | 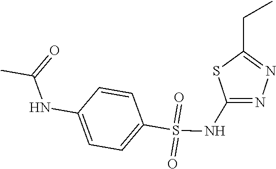 |
| | N-{4-[(5-ethyl-1,3,4-thiadiazol-2-yl)- |
| | sulfamoyl]phenyl}acetamide |
| RA22 | 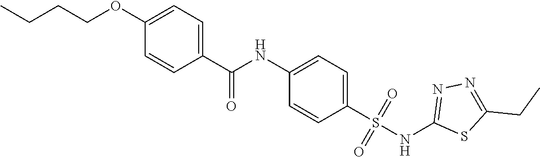 |
| | 4-butoxy-N-{4-[(5-ethyl-1,3,4-thiadiazol-2-yl)sulfamoyl]phenyl}benzamide |
| RA24 | 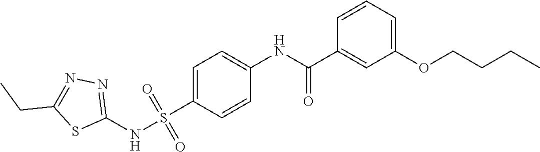 |
| | 3-butoxy-N-{4-[(5-ethyl-1,3,4-thiadiazol-2-yl)sulfamoyl]phenyl}benzamide |
| RA25 | 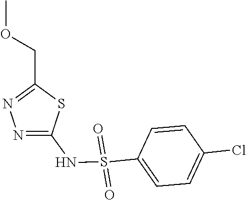 |
| | 4-chloro-N-[5-(methoxymethyl)-1,3,4- |
| | thiadiazol-2-yl]benzene-1-sulfonamide |
| RA28 | 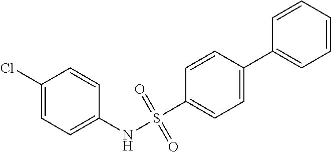 |
| | N-(4-chlorophenyl)-[1,1′-biphenyl]-4-sulfonamide |
| RA34 | 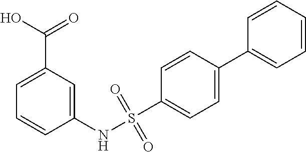 |
| | 3-{[1,1′-biphenyl]-4-sulfonamido}benzoic acid |
| RA13 | 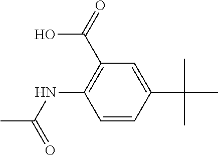 |
| | 5-tert-butyl-2-acetamidobenzoic acid |
| RA14 | 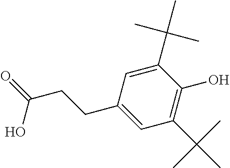 |
| | 3-(3,5-di-tert-butyl-4-hydroxyphenyl)- |
| | propanoic acid |
| RA15 | 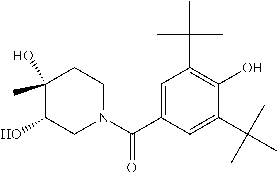 |
| | (3S,4R)-1-(3,5-di-tert-butyl-4-hydroxy- |
| | benzoyl)-4-methylpiperidine-3,4-diol |
| RA16 | 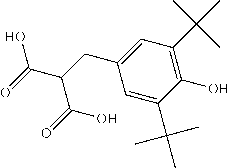 |
| | 2-[(3,5-di-tert-butyl-4-hydroxyphenyl)- |
| | methyl]propanedioic acid |
| RA126 | 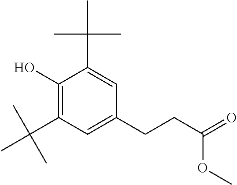 |
| | methyl 3-(3,5-di-tert-butyl-4-hydroxy- |
| | phenyl)propanoate |
| RA96 | 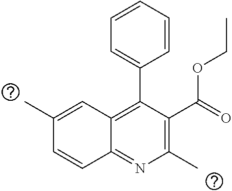 |
| | ethyl 2,6-dichloro-4-phenylquinoline- |
| | 3-carboxylate |
| RA97 | 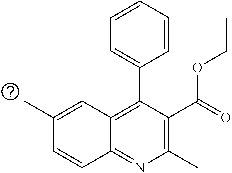 |
| | ethyl 6-chloro-2-methyl-4-phenyl- |
| | quinoline-3-carboxylate |
| RA108 | 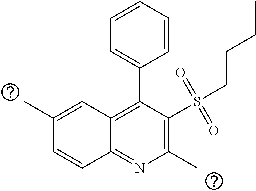 |
| | 3-(butane-1-sulfonyl)-6-chloro-4-phenyl- |
| | quinolin-2-ol |
| T744 | 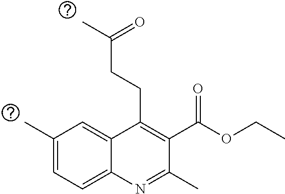 [SMILES: CCOC(=O)c1c(C)nc2ccc(C)cc2c1CCC(C)=O] |
| | 3-[6-chloro-3-(ethoxycarbonyl)-2-methyl- |
| | quinolin-4-yl]propanoic acid |
| T745 | 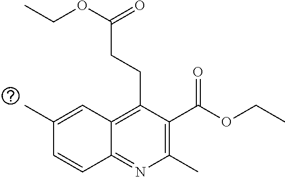 [SMILES: CCOC(=O)CCc1c(C(=O)OCC)c(C)nc2ccc(C)cc12] |
| | ethyl 6-chloro-4-(3-ethoxy-3-oxopropyl)-2- |
| | methylquinoline-3-carboxylate |
| T748 | 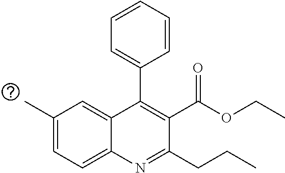 [SMILES: CCCc1nc2ccc(C)cc2c(-c2ccccc2)c1C(=O)OCC] |
| | ethyl 6-chloro-4-phenyl-2-propylquinoline- |
| | 3-carboxylate |
| T775 | 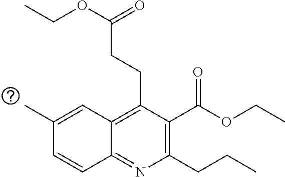 [SMILES: CCCc1nc2ccc(C)cc2c(CCC(=O)OCC)c1C(=O)OCC] |
| | ethyl 6-chloro-4-(3-ethoxy-3-oxopropyl)-2- |
| | propylquinoline-3-carboxylate |
| Acid Metabolite of T775 | 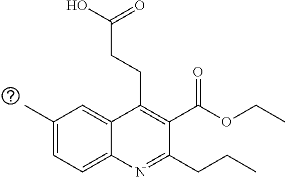 [SMILES: CCCc1nc2ccc(C)cc2c(CCC(=O)O)c1C(=O)OCC] |
| | 3-[6-chloro-3-(ethoxycarbonyl)-2-propyl- |
| | quinolin-4-yl]propanoic acid |
| T776 | 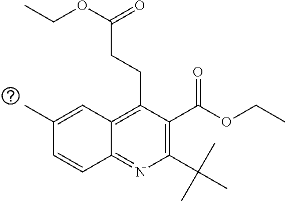 [SMILES: CCOC(=O)CCc1c(C(=O)OCC)c(C(C)(C)C)nc2ccc(C)cc12] |
| | ethyl 2-tert-butyl-6-chloro-4-(3-ethoxy-3- |
| | oxopropyl)quinoline-3-carboxylate |
| Acid Metabolite of T776 | 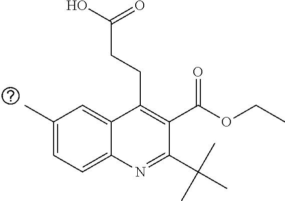 [SMILES: CCOC(=O)c1c(C(C)(C)C)nc2ccc(C)cc2c1CCC(=O)O] |
| | 3-[2-tert-butyl-6-chloro-3-(ethoxycarbonyl)- |
| | quinolin-4-yl]propanoic acid |

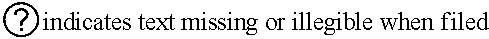

### Enumerated Embodiments

The following exemplary embodiments are provided, the numbering of which is not to be construed as designating levels of importance:

Embodiment 1 provides a method for promoting expansion, increasing viability, and/or altering functionality of a mammalian cell, the method comprising contacting the mammalian cell with at least one compound selected from the group consisting of formula (Ia), (Ib), (II), (III), and (IV), or a salt, solvate, prodrug, enantiomer, diastereoisomer or tautomer thereof:

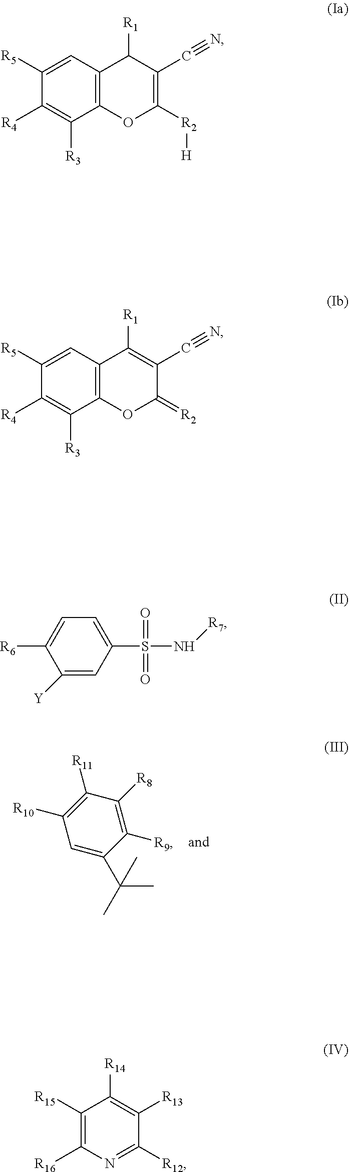

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of

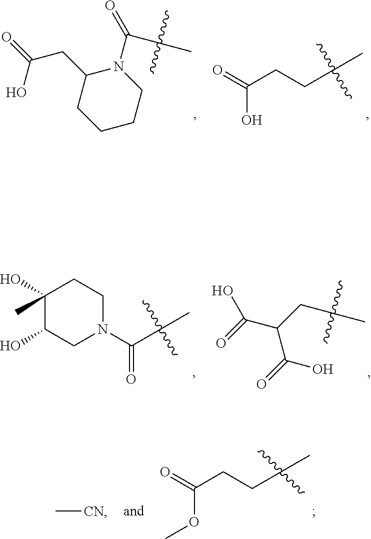

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 2 provides the method of Embodiment 1, wherein the compound is selected from the group consisting of:

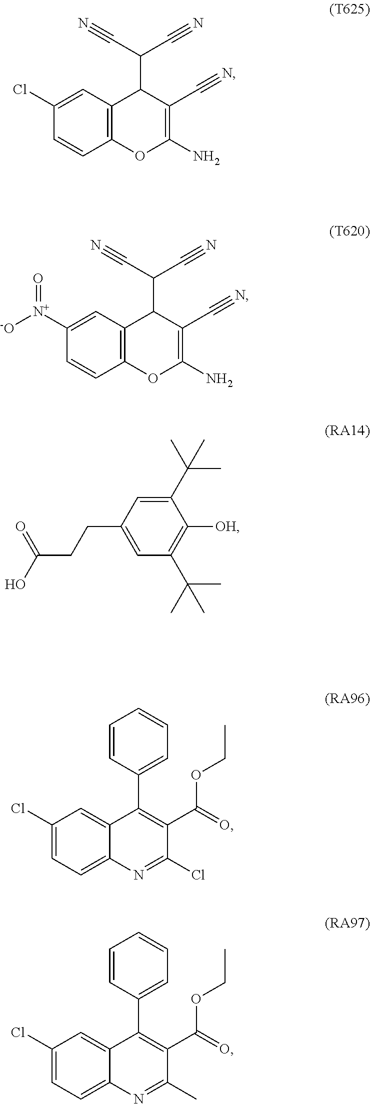

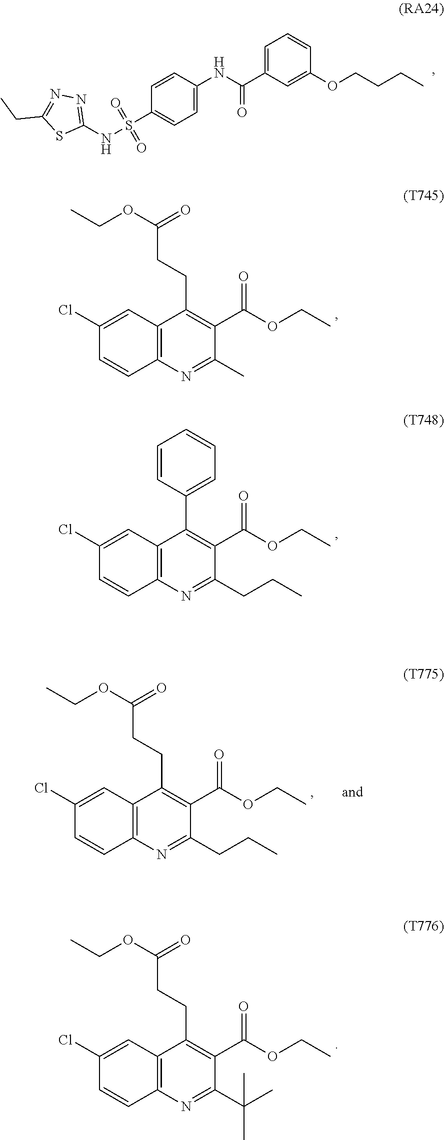 [SMILES: CCCCOc1cccc(C(=O)Nc2ccc(S(=O)(=O)Nc3nnc(CC)s3)cc2)c1 ; CCOC(=O)CCc1c(C(=O)OCC)c(C)nc2ccc(Cl)cc12 ; CCCc1nc2ccc(Cl)cc2c(-c2ccccc2)c1C(=O)OCC ; CCCc1nc2ccc(Cl)cc2c(CCC(=O)OCC)c1C(=O)OCC ; CCOC(=O)CCc1c(C(=O)OCC)c(C(C)(C)C)nc2ccc(Cl)cc12]

Embodiment 3 provides the method of Embodiment 1, wherein the compound is selected from the group consisting of:
2-(2-amino-6-chloro-3-cyano-4H-chromen-4-yl)propanedinitrile,
2-(2-amino-3-cyano-6-methoxy-4H-chromen-4-yl)propanedinitrile,
2-(2-amino-3-cyano-4H-chromen-4-yl)propanedinitrile,
2-(2-amino-6-bromo-3-cyano-4H-chromen-4-yl)propanedinitrile,
2-(2-amino-6,8-dichloro-3-cyano-4H-chromen-4-yl)propanedinitrile,
2-(2-amino-8-bromo-6-chloro-3-cyano-4H-chromen-4-yl)propanedinitrile,
2-(2-amino-6-chloro-3-cyano-8-methyl-4H-chromen-4-yl)propanedinitrile,
2-(2-amino-8-chloro-3-cyano-4H-chromen-4-yl)propanedinitrile,
2-(2-amino-7-chloro-3-cyano-4H-chromen-4-yl)propanedinitrile,
2-(2-amino-6-chloro-3-cyano-4H-chromen-4-yl)propanedinitrile, 2-(2-amino-8-bromo-3-cyano-4H-chromen-4-yl)propanedinitrile,
2-(2-amino-7-bromo-3-cyano-4H-chromen-4-yl)propanedinitrile,
2-(2-amino-3-cyano-8-methyl-4H-chromen-4-yl)propanedinitrile,
2-(2-amino-3-cyano-7-methyl-4H-chromen-4-yl)propanedinitrile,
2-(2-amino-3-cyano-6-methyl-4H-chromen-4-yl)propanedinitrile,
2-(2-amino-3-cyano-8-fluoro-4H-chromen-4-yl)propanedinitrile,
2-(2-amino-3-cyano-6-fluoro-4H-chromen-4-yl)propanedinitrile,
2-(2-amino-3-cyano-7-fluoro-4H-chromen-4-yl)propanedinitrile,
2-[2-amino-3-cyano-8-(trifluoromethyl)-4H-chromen-4-yl]propanedinitrile,
2-(2-amino-3-cyano-8-hydroxy-4H-chromen-4-yl)propanedinitrile, 2-(2-amino-3-cyano-6-nitro-4H-chromen-4-yl)propanedinitrile,
2-amino-3-cyano-4-(dicyanomethyl)-4H-chromene-8-carboxylic acid,
2-(2-amino-3-cyano-6-hydroxy-4H-chromen-4-yl)propanedinitrile,
2-(2-amino-6-chloro-3-cyano-8-hydroxy-4H-chromen-4-yl)propanedinitrile,
2-(2-amino-3-cyano-8-nitro-4H-chromen-4-yl)propanedinitrile,
2-(2-amino-6-chloro-3-cyano-4H-chromen-4-yl)propanedinitrile,
6-bromo-2-imino-2H-chromene-3-carbonitrile,
6-chloro-2-imino-2H-chromene-3-carbonitrile,
7-hydroxy-2-imino-2H-chromene-3-carbonitrile,
N-{4-[(5-ethyl-1,3,4-thiadiazol-2-yl)sulfamoyl]phenyl}acetamide,
N-{4-[(5-ethyl-1,3,4-thiadiazol-2-yl)sulfamoyl]phenyl}benzamide,
4-chloro-N-(5-ethyl-1,3,4-thiadiazol-2-yl)benzene-1-sulfonamide,
N-{4-[(5-methyl-1,3,4-thiadiazol-2-yl)sulfamoyl]phenyl}acetamide,
4-butoxy-N-{4-[(5-ethyl-1,3,4-thiadiazol-2-yl)sulfamoyl]phenyl}benzamide,
4-butyl-N-(5-methyl-1,3,4-thiadiazol-2-yl)benzene-1-sulfonamide,
3-butoxy-N-{4-[(5-ethyl-1,3,4-thiadiazol-2-yl)sulfamoyl]phenyl}benzamide,
4-chloro-N-[5-(methoxymethyl)-1,3,4-thiadiazol-2-yl]benzene-1-sulfonamide,
2-hexanoyl-N-(5-methyl-1,3,4-thiadiazol-2-yl)-1,2,3,4-tetrahydroisoquinoline-7-sulfonamide,
4-{[1,1′-biphenyl]-4-sulfonamido}benzoic acid,
N-(4-chlorophenyl)-[1,1′-biphenyl]-4-sulfonamide,
ethyl 4-{[1,1′-biphenyl]-4-sulfonamido}benzoate,
N-(5-chloropyridin-2-yl)-[1,1′-biphenyl]-4-sulfonamide,
N-(4-methyl-1,3-thiazol-2-yl)-[1,1′-biphenyl]-4-sulfonamide,
N-(5-methyl-1,2-oxazol-3-yl)-[1,1′-biphenyl]-4-sulfonamide,
N-{4-[(5-ethyl-1,3,4-thiadiazol-2-yl)sulfamoyl]phenyl}hexanamide,
3-{[1,1′-biphenyl]-4-sulfonamido}benzoic acid,
N-(pyridin-2-yl)-[1,1′-biphenyl]-4-sulfonamide,
2-[4-(3,5-di-tert-butyl-4-hydroxybenzoyl)morpholin-3-yl]acetic acid,
5-tert-butyl-2-acetamidobenzoic acid,
3-(3,5-di-tert-butyl-4-hydroxyphenyl)propanoic acid,
(3S,4R)-1-(3,5-di-tert-butyl-4-hydroxybenzoyl)-4-methylpiperidine-3,4-diol,
2-[(3,5-di-tert-butyl-4-hydroxyphenyl)methyl]propanedioic acid,
3,5-di-tert-butyl-4-hydroxybenzonitrile,
methyl 3-(3,5-di-tert-butyl-4-hydroxyphenyl)propanoate,
ethyl 2-amino-3-cyano-4-(2-methylphenyl)-5,6,7,8-tetrahydro-1,6-naphthyridine-6-carboxylate,
2-amino-6-cyclopropanecarbonyl-4-[5-(hydroxymethyl)furan-2-yl]-5,6,7,8-tetrahydro-1,6-naphthyridine-3-carbonitrile,
ethyl 2-amino-3-cyano-4-[5-(hydroxymethyl)furan-2-yl]-5,6,7,8-tetrahydroquinoline-6-carboxylate,
ethyl 2,6-dichloro-4-phenylquinoline-3-carboxylate,
ethyl 2,6-dibromo-4-phenylquinoline-3-carboxylate,
ethyl 2,6-dinitro-4-phenylquinoline-3-carboxylate,
ethyl 5-chloro-2-methyl-4-phenylquinoline-3-carboxylate,
ethyl 6-chloro-2-methyl-4-phenylquinoline-3-carboxylate,
ethyl 7-chloro-2-methyl-4-phenylquinoline-3-carboxylate,
ethyl 8-chloro-2-methyl-4-phenylquinoline-3-carboxylate,
ethyl 5-bromo-2-methyl-4-phenylquinoline-3-carboxylate,
ethyl 6-bromo-2-methyl-4-phenylquinoline-3-carboxylate,
ethyl 7-bromo-2-methyl-4-phenylquinoline-3-carboxylate,
ethyl 8-bromo-2-methyl-4-phenylquinoline-3-carboxylate,
ethyl 5-nitro-2-methyl-4-phenylquinoline-3-carboxylate,
ethyl 6-nitro-2-methyl-4-phenylquinoline-3-carboxylate,
ethyl 7-nitro-2-methyl-4-phenylquinoline-3-carboxylate,
ethyl 8-nitro-2-methyl-4-phenylquinoline-3-carboxylate,
ethyl 5-chloro-4-(morpholin-4-yl)quinoline-3-carboxylate,
ethyl 6-chloro-4-(morpholin-4-yl)quinoline-3-carboxylate,
ethyl 7-chloro-4-(morpholin-4-yl)quinoline-3-carboxylate,
ethyl 8-chloro-4-(morpholin-4-yl)quinoline-3-carboxylate,
ethyl 5-bromo-4-(morpholin-4-yl)quinoline-3-carboxylate,
ethyl 6-bromo-4-(morpholin-4-yl)quinoline-3-carboxylate,
ethyl 7-bromo-4-(morpholin-4-yl)quinoline-3-carboxylate,
ethyl 8-bromo-4-(morpholin-4-yl)quinoline-3-carboxylate,
ethyl 5-nitro-4-(morpholin-4-yl)quinoline-3-carboxylate,
ethyl 6-nitro-4-(morpholin-4-yl)quinoline-3-carboxylate,
ethyl 7-nitro-4-(morpholin-4-yl)quinoline-3-carboxylate,
ethyl 8-nitro-4-(morpholin-4-yl)quinoline-3-carboxylate,
3-(butane-1-sulfonyl)-6-chloro-4-phenylquinolin-2-ol,
2-amino-4-(5-cyano-1,2-dimethyl-1H-pyrrol-3-yl)-6-cyclopropanecarbonyl-5,6,7,8-tetrahydro-1,6-naphthyridine-3-carbonitrile,
2-amino-4-(5-chlorothiophen-2-yl)-6-cyclopropanecarbonyl-5,6,7,8-tetrahydro-1,6-naphthyridine-3-carbonitrile,
methyl 3-(2-amino-6-butanoyl-3-cyano-5,6,7,8-tetrahydro-1,6-naphthyridin-4-yl)benzoate,
ethyl 2-amino-3-cyano-4-(5-cyano-1-methyl-1H-pyrrol-3-yl)-5,6,7,8-tetrahydroquinoline-6-carboxylate,
ethyl 2-amino-3-cyano-4-(4-hydroxy-2-methoxyphenyl)-5,6,7,8-tetrahydroquinoline-6-carboxylate,
3-[5-chloro-3-(ethoxycarbonyl)-2-methylquinolin-4-yl]propanoic acid,
3-[6-chloro-3-(ethoxycarbonyl)-2-methylquinolin-4-yl]propanoic acid,
3-[7-chloro-3-(ethoxycarbonyl)-2-methylquinolin-4-yl]propanoic acid,
3-[8-chloro-3-(ethoxycarbonyl)-2-methylquinolin-4-yl]propanoic acid,
3-[5-bromo-3-(ethoxycarbonyl)-2-methylquinolin-4-yl]propanoic acid,
3-[6-bromo-3-(ethoxycarbonyl)-2-methylquinolin-4-yl]propanoic acid,
3-[7-bromo-3-(ethoxycarbonyl)-2-methylquinolin-4-yl]propanoic acid,
3-[8-bromo-3-(ethoxycarbonyl)-2-methylquinolin-4-yl]propanoic acid,
3-[5-nitro-3-(ethoxycarbonyl)-2-methylquinolin-4-yl]propanoic acid,
3-[6-nitro-3-(ethoxycarbonyl)-2-methylquinolin-4-yl]propanoic acid,
3-[7-nitro-3-(ethoxycarbonyl)-2-methylquinolin-4-yl]propanoic acid,
3-[8-nitro-3-(ethoxycarbonyl)-2-methylquinolin-4-yl]propanoic acid,
ethyl 5-chloro-4-(3-ethoxy-3-oxopropyl)-2-methylquinoline-3-carboxylate,
ethyl 6-chloro-4-(3-ethoxy-3-oxopropyl)-2-methylquinoline-3-carboxylate,
ethyl 7-chloro-4-(3-ethoxy-3-oxopropyl)-2-methylquinoline-3-carboxylate,
ethyl 8-chloro-4-(3-ethoxy-3-oxopropyl)-2-methylquinoline-3-carboxylate,
ethyl 5-bromo-4-(3-ethoxy-3-oxopropyl)-2-methylquinoline-3-carboxylate,
ethyl 6-bromo-4-(3-ethoxy-3-oxopropyl)-2-methylquinoline-3-carboxylate,
ethyl 7-bromo-4-(3-ethoxy-3-oxopropyl)-2-methylquinoline-3-carboxylate,
ethyl 8-bromo-4-(3-ethoxy-3-oxopropyl)-2-methylquinoline-3-carboxylate,
ethyl 4-(3-ethoxy-3-oxopropyl)-2-methyl-5-nitroquinoline-3-carboxylate,
ethyl 4-(3-ethoxy-3-oxopropyl)-2-methyl-6-nitroquinoline-3-carboxylate
ethyl 4-(3-ethoxy-3-oxopropyl)-2-methyl-7-nitroquinoline-3-carboxylate,
ethyl 4-(3-ethoxy-3-oxopropyl)-2-methyl-8-nitroquinoline-3-carboxylate,
ethyl 5-chloro-4-phenyl-2-propylquinoline-3-carboxylate,
ethyl 6-chloro-4-phenyl-2-propylquinoline-3-carboxylate,
ethyl 7-chloro-4-phenyl-2-propylquinoline-3-carboxylate,
ethyl 8-chloro-4-phenyl-2-propylquinoline-3-carboxylate,
ethyl 5-bromo-4-phenyl-2-propylquinoline-3-carboxylate,
ethyl 6-bromo-4-phenyl-2-propylquinoline-3-carboxylate,
ethyl 7-bromo-4-phenyl-2-propylquinoline-3-carboxylate,
ethyl 8-bromo-4-phenyl-2-propylquinoline-3-carboxylate,
ethyl 5-nitro-4-phenyl-2-propylquinoline-3-carboxylate,
ethyl 6-nitro-4-phenyl-2-propylquinoline-3-carboxylate,
ethyl 7-nitro-4-phenyl-2-propylquinoline-3-carboxylate,
ethyl 8-nitro-4-phenyl-2-propylquinoline-3-carboxylate,
3-[5-chloro-3-(ethoxycarbonyl)-2-propylquinolin-4-yl]propanoic acid,
3-[6-chloro-3-(ethoxycarbonyl)-2-propylquinolin-4-yl]propanoic acid,
3-[7-chloro-3-(ethoxycarbonyl)-2-propylquinolin-4-yl]propanoic acid,
3-[8-chloro-3-(ethoxycarbonyl)-2-propylquinolin-4-yl]propanoic acid,
3-[5-bromo-3-(ethoxycarbonyl)-2-propylquinolin-4-yl]propanoic acid,
3-[6-bromo-3-(ethoxycarbonyl)-2-propylquinolin-4-yl]propanoic acid,
3-[7-bromo-3-(ethoxycarbonyl)-2-propylquinolin-4-yl]propanoic acid,
3-[8-bromo-3-(ethoxycarbonyl)-2-propylquinolin-4-yl]propanoic acid,
3-[5-nitro-3-(ethoxycarbonyl)-2-propylquinolin-4-yl]propanoic acid,
3-[6-nitro-3-(ethoxycarbonyl)-2-propylquinolin-4-yl]propanoic acid,
3-[7-nitro-3-(ethoxycarbonyl)-2-propylquinolin-4-yl]propanoic acid,
3-[8-nitro-3-(ethoxycarbonyl)-2-propylquinolin-4-yl]propanoic acid,
ethyl 5-chloro-4-(3-ethoxy-3-oxopropyl)-2-propylquinoline-3-carboxylate,
ethyl 6-chloro-4-(3-ethoxy-3-oxopropyl)-2-propylquinoline-3-carboxylate,
ethyl 7-chloro-4-(3-ethoxy-3-oxopropyl)-2-propylquinoline-3-carboxylate,
ethyl 8-chloro-4-(3-ethoxy-3-oxopropyl)-2-propylquinoline-3-carboxylate,
ethyl 5-bromo-4-(3-ethoxy-3-oxopropyl)-2-propylquinoline-3-carboxylate,
ethyl 6-bromo-4-(3-ethoxy-3-oxopropyl)-2-propylquinoline-3-carboxylate,
ethyl 7-bromo-4-(3-ethoxy-3-oxopropyl)-2-propylquinoline-3-carboxylate,
ethyl 8-bromo-4-(3-ethoxy-3-oxopropyl)-2-propylquinoline-3-carboxylate,
ethyl 4-(3-ethoxy-3-oxopropyl)-5-nitro-2-propylquinoline-3-carboxylate,
ethyl 4-(3-ethoxy-3-oxopropyl)-6-nitro-2-propylquinoline-3-carboxylate,
ethyl 4-(3-ethoxy-3-oxopropyl)-7-nitro-2-propylquinoline-3-carboxylate,
ethyl 4-(3-ethoxy-3-oxopropyl)-8-nitro-2-propylquinoline-3-carboxylate,
3-[2-tert-butyl-5-chloro-3-(ethoxycarbonyl)quinolin-4-yl]propanoic acid,
3-[2-tert-butyl-6-chloro-3-(ethoxycarbonyl)quinolin-4-yl]propanoic acid,
3-[2-tert-butyl-7-chloro-3-(ethoxycarbonyl)quinolin-4-yl]propanoic acid,
3-[2-tert-butyl-8-chloro-3-(ethoxycarbonyl)quinolin-4-yl]propanoic acid,
3-[2-tert-butyl-5-bromo-3-(ethoxycarbonyl)quinolin-4-yl]propanoic acid,
3-[2-tert-butyl-6-bromo-3-(ethoxycarbonyl)quinolin-4-yl]propanoic acid,
3-[2-tert-butyl-7-bromo-3-(ethoxycarbonyl)quinolin-4-yl]propanoic acid,
3-[2-tert-butyl-8-bromo-3-(ethoxycarbonyl)quinolin-4-yl]propanoic acid,
3-[2-tert-butyl-5-nitro-3-(ethoxycarbonyl)quinolin-4-yl]propanoic acid,
3-[2-tert-butyl-6-nitro-3-(ethoxycarbonyl)quinolin-4-yl]propanoic acid,
3-[2-tert-butyl-7-nitro-3-(ethoxycarbonyl)quinolin-4-yl]propanoic acid,
3-[2-tert-butyl-8-nitro-3-(ethoxycarbonyl)quinolin-4-yl]propanoic acid,
ethyl 2-(tert-butyl)-5-chloro-4-(3-ethoxy-3-oxopropyl)quinoline-3-carboxylate,
ethyl 2-(tert-butyl)-6-chloro-4-(3-ethoxy-3-oxopropyl)quinoline-3-carboxylate,
ethyl 2-(tert-butyl)-7-chloro-4-(3-ethoxy-3-oxopropyl)quinoline-3-carboxylate
ethyl 2-(tert-butyl)-8-chloro-4-(3-ethoxy-3-oxopropyl)quinoline-3-carboxylate,
ethyl 5-bromo-2-(tert-butyl)-4-(3-ethoxy-3-oxopropyl)quinoline-3-carboxylate,
ethyl 6-bromo-2-(tert-butyl)-4-(3-ethoxy-3-oxopropyl)quinoline-3-carboxylate,
ethyl 7-bromo-2-(tert-butyl)-4-(3-ethoxy-3-oxopropyl)quinoline-3-carboxylate,
ethyl 8-bromo-2-(tert-butyl)-4-(3-ethoxy-3-oxopropyl)quinoline-3-carboxylate,
ethyl 2-(tert-butyl)-4-(3-ethoxy-3-oxopropyl)-5-nitroquinoline-3-carboxylate,
ethyl 2-(tert-butyl)-4-(3-ethoxy-3-oxopropyl)-6-nitroquinoline-3-carboxylate,
ethyl 2-(tert-butyl)-4-(3-ethoxy-3-oxopropyl)-7-nitroquinoline-3-carboxylate, and
ethyl 2-(tert-butyl)-4-(3-ethoxy-3-oxopropyl)-8-nitroquinoline-3-carboxylate.

Embodiment 4 provides the method of any one of Embodiments 1-3, wherein the mammalian cell is an immune cell.

Embodiment 5 provides the method of Embodiment 4, wherein the immune cell is an innate and/or adaptive immune cell selected from the group consisting of hematopoietic progenitor cell, B lymphocyte, T lymphocyte, natural killer cell, and myeloid lineage cell.

Embodiment 6 provides the method of any one of Embodiments 1-5, wherein the mammalian cell is a keratinocyte.

Embodiment 7 provides the method of any one of Embodiments 1-6, wherein the at least one compound is an Akt agonist with selectivity for a single isoform of Akt kinase selected from the group consisting of Akt1, Akt2, and Akt3.

Embodiment 8 provides the method of any one of Embodiments 1-7, wherein the at least one compound enhances Akt kinase phosphatase resistance.

Embodiment 9 provides the method of any one of Embodiments 1-8, wherein the mammalian cell is contacted ex vivo with the at least one compound.

Embodiment 10 provides the method of any one of Embodiments 1-9, wherein the contacted mammalian cell is reintroduced in vivo.

Embodiment 11 provides the method of any one of Embodiments 1-10, wherein the mammalian cell is present in vivo.

Embodiment 12 provides a method of inhibiting, treating, and/or preventing symptoms or complications of a metabolic disease, pulmonary condition, diabetes, cardiovascular condition, and/or neurological/neurodegenerative disease in a subject, the method comprising administering to the subject a therapeutically effective amount of the at least one compound of formula (Ia), (Ib), (II), (III), and (IV), or a salt, solvate, prodrug, enantiomer, diastereoisomer or tautomer thereof:

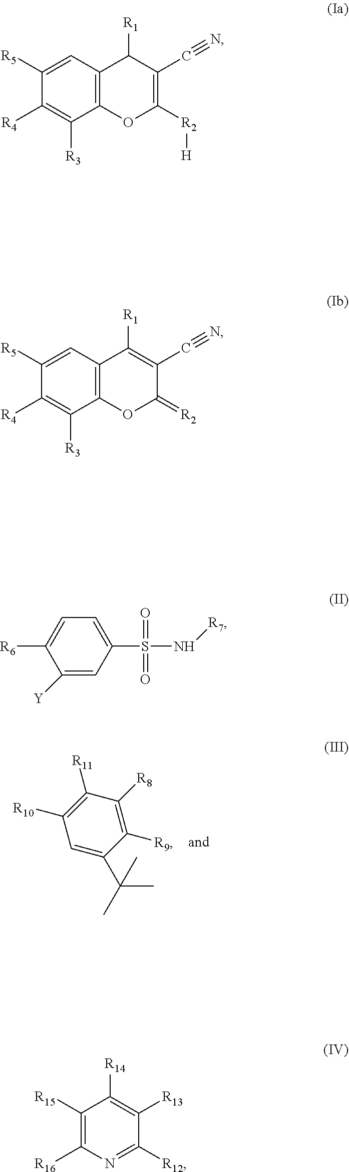

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of:

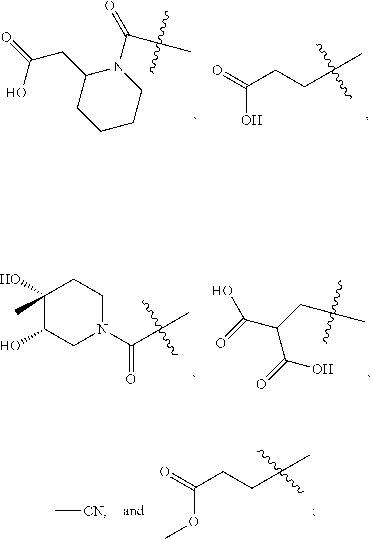

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 13 provides the method of Embodiment 12, wherein the cardiovascular condition comprises myocardial infarction or stroke.

Embodiment 14 provides the method of Embodiment 12 or 13, wherein the pulmonary condition comprises acute lung injury or ventilator-induced lung injury.

Embodiment 15 provides the method of any one of Embodiments 12-14, wherein the metabolic disease comprises an acute metabolic disease.

Embodiment 16 provides a method of treating cancer in a subject, the method comprising:
(a) administering to the subject a therapeutically effective amount of the at least one compound of formula (Ia), (Ib), (II), (III), and (IV), or a salt, solvate, prodrug, enantiomer, diastereoisomer or tautomer thereof; or
(b) administering to the subject a therapeutically effective amount of cells pre-treated with the at least one compound of formula (Ia), (Ib), (II), (III), and (IV), or a salt, solvate, prodrug, enantiomer, diastereoisomer or tautomer thereof:

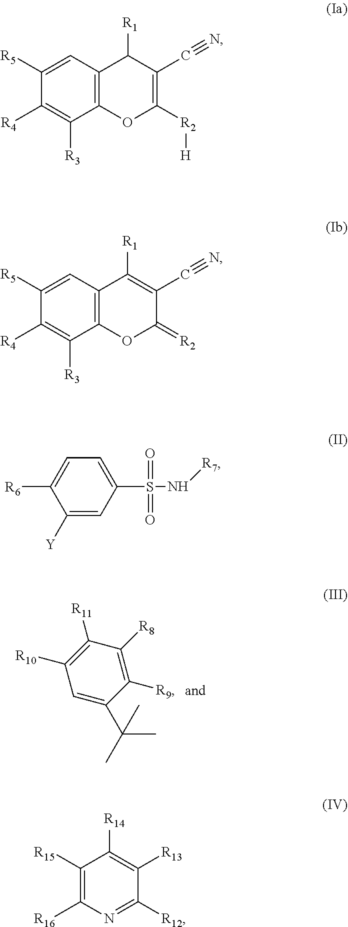

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of:

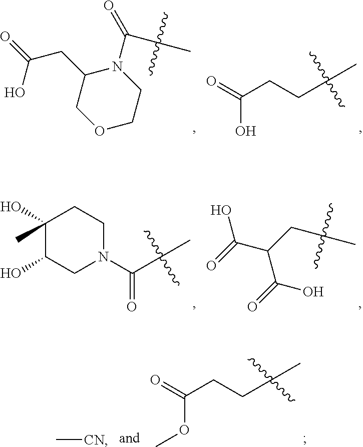

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 17 provides a method of promoting tissue replacement, promoting tissue regeneration, and/or treating degenerative diseases in a subject, the method comprising:
(a) administering to the subject a therapeutically effective amount of the at least one compound of formula (Ia), (Ib), (II), (III), and (IV), or a salt, solvate, prodrug, enantiomer, diastereoisomer or tautomer thereof; or
(b) administering to the subject a therapeutically effective amount of cells pre-treated with the at least one compound of formula (Ia), (Ib), (II), (III), and (IV), or a salt, solvate, prodrug, enantiomer, diastereoisomer or tautomer thereof:

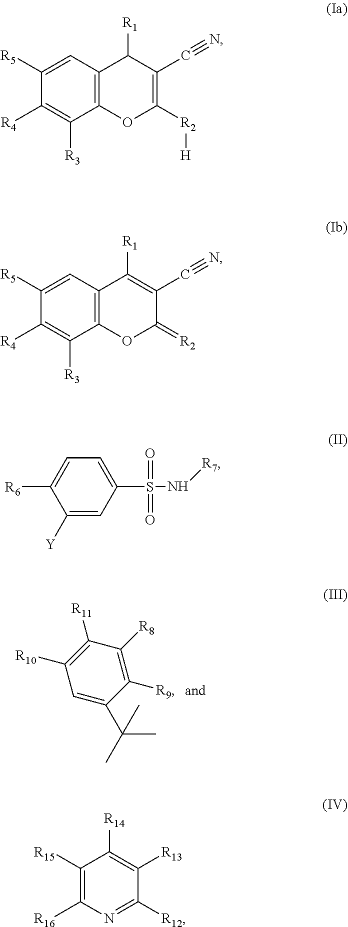

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of:

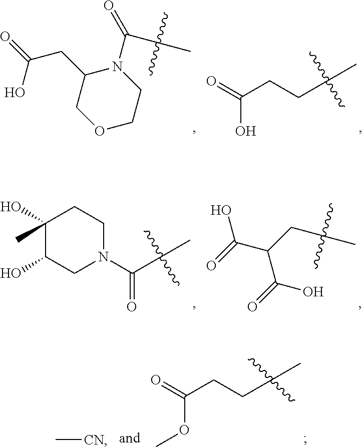

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 18 provides a method of treating or healing a skin condition and/or a wound in a subject, the method comprising topically treating the skin and/or wound with a therapeutically effective amount of the at least one compound of formula (Ia), (Ib), (II), (III), and (IV), or a salt, solvate, prodrug, enantiomer, diastereoisomer or tautomer thereof:

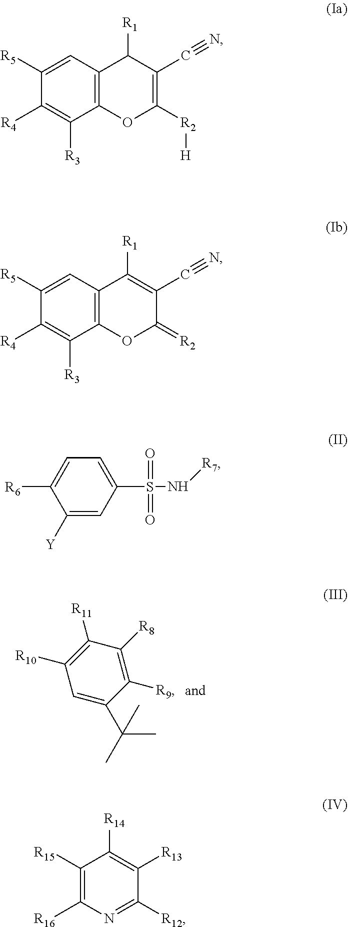

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of

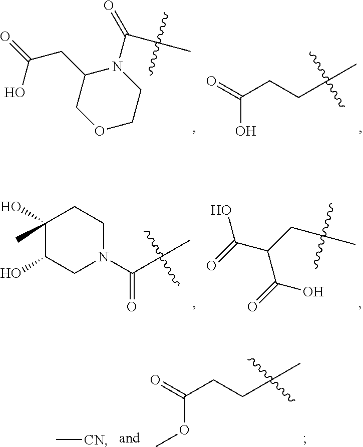

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 19 provides the method of any one of Embodiments 12-18, wherein the administration duration is equal to or less than 14 days.

Embodiment 20 provides the method of any one of Embodiments 12-19, wherein the administration does not cause any significant deleterious or unwanted cell multiplication in the subject.

Embodiment 21 provides the method of any one of Embodiments 12-20, which does not cause significant systemic exposure of the at least one compound in the subject.

Embodiment 22 provides the method of any one of Embodiments 12-21, wherein the treatment enhances formation of new connective tissue and/or microscopic blood vessels in the subject.

Embodiment 23 provides the method of any one of Embodiments 12-22, wherein the subject is a mammal.

Embodiment 24 provides the method of Embodiment 23, wherein the mammal is a human.

Embodiment 25 provides a method of inhibiting dephosphorylation of Akt1, Akt2 and/or Akt3, the method comprising contacting the pleckstrin homology domains of Akt1, Akt2 and/or Akt3 with the at least one compound of formula (Ia), (Ib), (II), (III), and (IV), or a salt, solvate, prodrug, enantiomer, diastereoisomer or tautomer thereof:

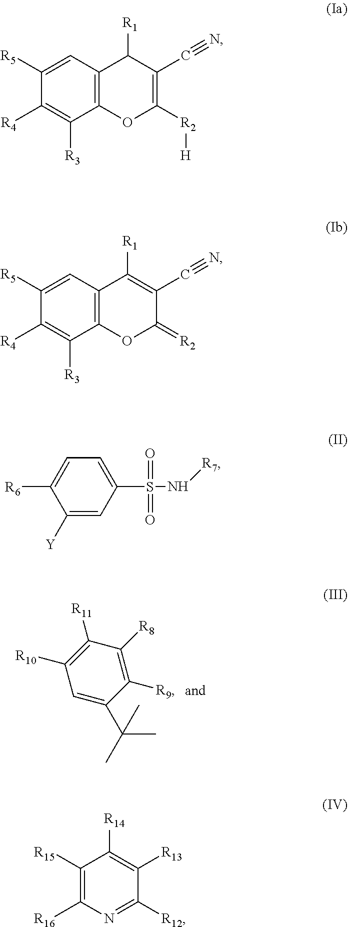

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of:

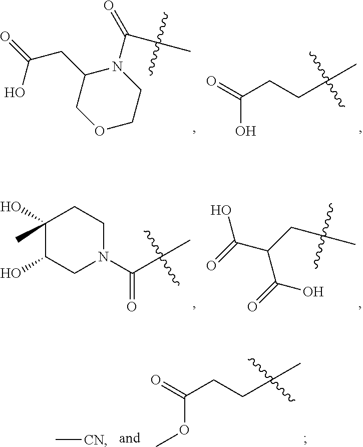

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 26 provides a method of (a) increasing phosphorylation states of Akt1, Akt2 and/or Akt3 in a human cell, (b) increasing catalytic activities of Akt1, Akt2 and/or Akt3 in a human cell, and/or (c) increasing phosphorylation of substrates of Akt1, Akt2 or Akt3 in a human cell, the method comprising contacting the human cell with of the at least one compound of formula (Ia), (Ib), (II), (III), and (IV), or a salt, solvate, prodrug, enantiomer, diastereoisomer or tautomer thereof:

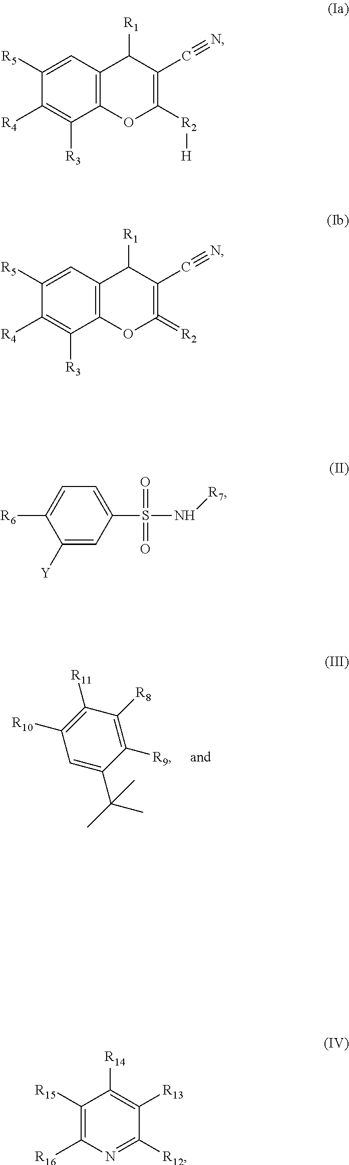

wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
   wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
   wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
   wherein the heteroaryl is selected from the group consisting of thiadiazolyl, thiazolyl, oxazolyl, diazolyl, imidazolyl, triazinyl, thiazolyl, isothiazolyl, and pyridinyl, and
   wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
   wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of:

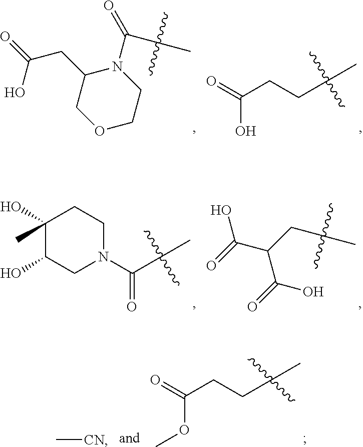

R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
   wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
   wherein n is an integer from 1-6,
   wherein the heteroaryl is selected from the group consisting of furanyl, pyrrolyl, and thiofuranyl, and
   wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
   wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 27 provides a compound of formula (Va) or (Vb), or a salt, solvate, prodrug, enantiomer, diastereoisomer or tautomer thereof:

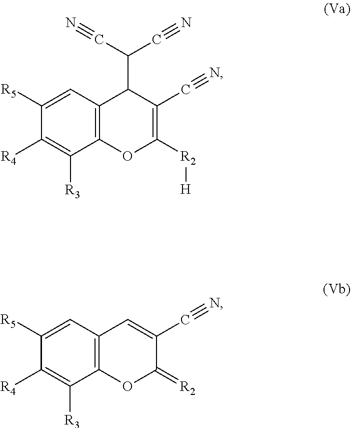

wherein:
R₂ is NH;
R₃ is selected from the group consisting of hydrogen, optionally substituted C₁-C₆ alkyl, optionally substituted C₃-C₈ cycloalkyl, optionally substituted C₂-C₆ alkenyl, optionally substituted C₂-C₆ alkynyl, hydroxyl, optionally substituted C₁-C₆ alkoxy, halogen, haloalkyl, nitro, and —C(═O)OR′, wherein R′ is selected from the group consisting of H, optionally substituted C₁-C₆ alkyl, optionally substituted C₃-C₈ cycloalkyl, optionally substituted C₂-C₈ alkenyl, and optionally substituted C₂-C₈ alkynyl,
   wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₄ is selected from the group consisting of hydrogen, halogen, optionally substituted C₁-C₆ alkyl, optionally substituted C₃-C₈ alkyl, optionally substituted C₂-C₈ alkenyl, optionally substituted C₂-C₈ alkynyl, optionally substituted C₁-C₆ alkoxy, and hydroxyl,
   wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₅ is selected from the group consisting of hydrogen, halogen, optionally substituted C₁-C₆ alkyl, optionally substituted C₃-C₃ cycloalkyl, optionally substituted C₂-C₈ alkenyl, optionally substituted C₂-C₈ alkynyl, nitro, hydroxyl, and optionally substituted C₁-C₆ alkoxy,
   wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

Embodiment 28 provides a pharmaceutical composition comprising at least one pharmaceutically acceptable carrier and at least one compound of Embodiment 27.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. A method for:
i. promoting expansion, increasing viability, or altering functionality of a mammalian cell,
ii. inhibiting, treating, or preventing symptoms or complications of a metabolic disease, pulmonary condition, diabetes, cardiovascular condition, or neurological/neurodegenerative disease in a mammalian cell;
iii. inhibiting dephosphorylation of Akt1, Akt2 or Akt3 in a mammalian cell: or
iv. increasing phosphorylation states of Akt1, Akt2 or Akt3 in a mammalian cell, increasing catalytic activities of Akt1, Akt2 or Akt3 in a mammalian cell, or increasing phosphorylation of substrates of Akt1, Akt2 or Akt3 in a mammalian cell,
the method comprising contacting the mammalian cell with at least one compound selected from the group consisting of formula (Ia), (Ib), (II), (III), and (IV), or a salt, solvate, prodrug, enantiomer, diastereoisomer or tautomer thereof: 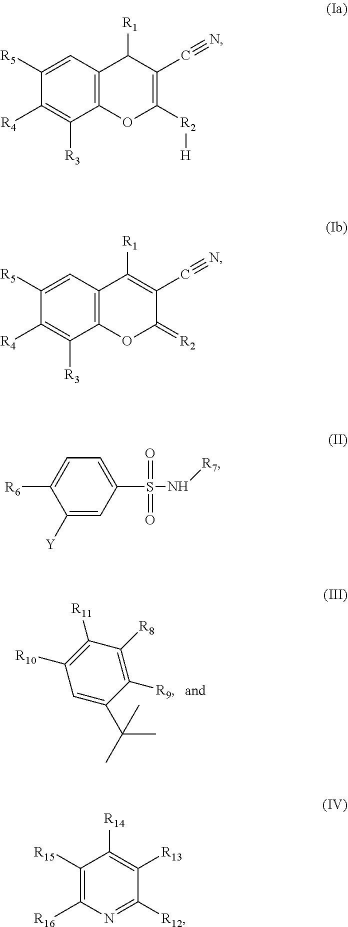
wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
wherein the heteroaryl is selected from the group consisting of thiadiazolyl, thiazolyl, oxazolyl, diazolyl, imidazolyl, triazinyl, thiazolyl, isothiazolyl, and pyridinyl, and
wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of: 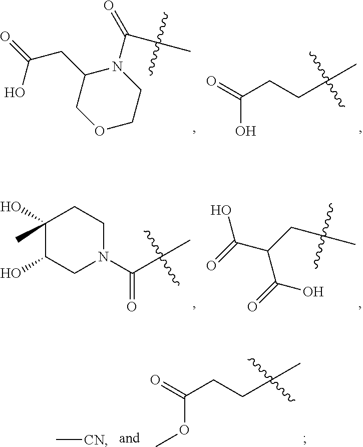
R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
wherein n is an integer from 1-6,
wherein the heteroaryl is selected from the group consisting of furanyl, pyrrolyl, and thiofuranyl, and
wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

2. The method of claim 1, wherein the compound is selected from the group consisting of:

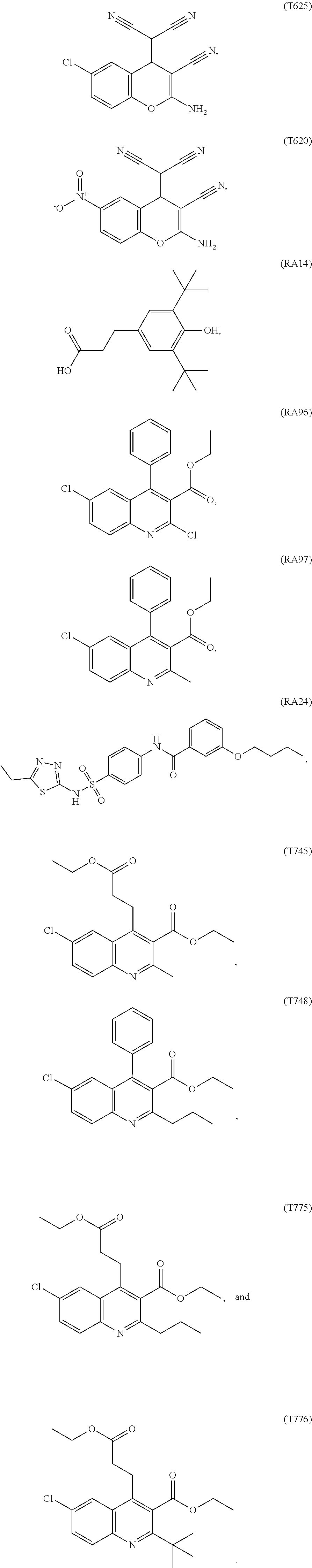

3. The method of claim 1, wherein the compound is selected from the group consisting of:
2-(2-amino-6-chloro-3-cyano-4H-chromen-4-yl)propanedinitrile,
2-(2-amino-3-cyano-6-methoxy-4H-chromen-4-yl)propanedinitrile,
2-(2-amino-3-cyano-4H-chromen-4-yl)propanedinitrile,
2-(2-amino-6-bromo-3-cyano-4H-chromen-4-yl)propanedinitrile,
2-(2-amino-6,8-dichloro-3-cyano-4H-chromen-4-yl)propanedinitrile,
2-(2-amino-8-bromo-6-chloro-3-cyano-4H-chromen-4-yl)propanedinitrile,
2-(2-amino-6-chloro-3-cyano-8-methyl-4H-chromen-4-yl)propanedinitrile,
2-(2-amino-8-chloro-3-cyano-4H-chromen-4-yl)propanedinitrile,
2-(2-amino-7-chloro-3-cyano-4H-chromen-4-yl)propanedinitrile,
2-(2-amino-6-chloro-3-cyano-4H-chromen-4-yl)propanedinitrile, 2-(2-amino-8-bromo-3-cyano-4H-chromen-4-yl)propanedinitrile,
2-(2-amino-7-bromo-3-cyano-4H-chromen-4-yl)propanedinitrile,
2-(2-amino-3-cyano-8-methyl-4H-chromen-4-yl)propanedinitrile,
2-(2-amino-3-cyano-7-methyl-4H-chromen-4-yl)propanedinitrile,
2-(2-amino-3-cyano-6-methyl-4H-chromen-4-yl)propanedinitrile,
2-(2-amino-3-cyano-8-fluoro-4H-chromen-4-yl)propanedinitrile,
2-(2-amino-3-cyano-6-fluoro-4H-chromen-4-yl)propanedinitrile,
2-(2-amino-3-cyano-7-fluoro-4H-chromen-4-yl)propanedinitrile,
2-[2-amino-3-cyano-8-(trifluoromethyl)-4H-chromen-4-yl]propanedinitrile,
2-(2-amino-3-cyano-8-hydroxy-4H-chromen-4-yl)propanedinitrile, 2-(2-amino-3-cyano-6-nitro-4H-chromen-4-yl)propanedinitrile,
2-amino-3-cyano-4-(dicyanomethyl)-4H-chromene-8-carboxylic acid,
2-(2-amino-3-cyano-6-hydroxy-4H-chromen-4-yl)propanedinitrile,
2-(2-amino-6-chloro-3-cyano-8-hydroxy-4H-chromen-4-yl)propanedinitrile,
2-(2-amino-3-cyano-8-nitro-4H-chromen-4-yl)propanedinitrile,
2-(2-amino-6-chloro-3-cyano-4H-chromen-4-yl)propanedinitrile,
6-bromo-2-imino-2H-chromene-3-carbonitrile,
6-chloro-2-imino-2H-chromene-3-carbonitrile,
7-hydroxy-2-imino-2H-chromene-3-carbonitrile,
N-{4-[(5-ethyl-1,3,4-thiadiazol-2-yl)sulfamoyl]phenyl}acetamide,
N-{4-[(5-ethyl-1,3,4-thiadiazol-2-yl)sulfamoyl]phenyl}benzamide,
4-chloro-N-(5-ethyl-1,3,4-thiadiazol-2-yl)benzene-1-sulfonamide,
N-{4-[(5-methyl-1,3,4-thiadiazol-2-yl)sulfamoyl]phenyl}acetamide,
4-butoxy-N-{4-[(5-ethyl-1,3,4-thiadiazol-2-yl)sulfamoyl]phenyl}benzamide,
4-butyl-N-(5-methyl-1,3,4-thiadiazol-2-yl)benzene-1-sulfonamide,
3-butoxy-N-{4-[(5-ethyl-1,3,4-thiadiazol-2-yl)sulfamoyl]phenyl}benzamide,
4-chloro-N-[5-(methoxymethyl)-1,3,4-thiadiazol-2-yl]benzene-1-sulfonamide,
2-hexanoyl-N-(5-methyl-1,3,4-thiadiazol-2-yl)-1,2,3,4-tetrahydroisoquinoline-7-sulfonamide,
4-{[1,1′-biphenyl]-4-sulfonamido}benzoic acid,
N-(4-chlorophenyl)-[1,1′-biphenyl]-4-sulfonamide,
ethyl 4-{[1,1′-biphenyl]-4-sulfonamido}benzoate,
N-(5-chloropyridin-2-yl)-[1,1′-biphenyl]-4-sulfonamide,
N-(4-methyl-1,3-thiazol-2-yl)-[1,1′-biphenyl]-4-sulfonamide,
N-(5-methyl-1,2-oxazol-3-yl)-[1,1′-biphenyl]-4-sulfonamide,
N-{4-[(5-ethyl-1,3,4-thiadiazol-2-yl)sulfamoyl]phenyl}hexanamide,
3-{[1,1′-biphenyl]-4-sulfonamido}benzoic acid,
N-(pyridin-2-yl)-[1,1′-biphenyl]-4-sulfonamide,
2-[4-(3,5-di-tert-butyl-4-hydroxybenzoyl)morpholin-3-yl]acetic acid,
5-tert-butyl-2-acetamidobenzoic acid,
3-(3,5-di-tert-butyl-4-hydroxyphenyl)propanoic acid,
(3S,4R)-1-(3,5-di-tert-butyl-4-hydroxybenzoyl)-4-methylpiperidine-3,4-diol,
2-[(3,5-di-tert-butyl-4-hydroxyphenyl)methyl]propanedioic acid,
3,5-di-tert-butyl-4-hydroxybenzonitrile,
methyl 3-(3,5-di-tert-butyl-4-hydroxyphenyl)propanoate,
ethyl 2-amino-3-cyano-4-(2-methylphenyl)-5,6,7,8-tetrahydro-1,6-naphthyridine-6-carboxylate,
2-amino-6-cyclopropanecarbonyl-4-[5-(hydroxymethyl)furan-2-yl]-5,6,7,8-tetrahydro-1,6-naphthyridine-3-carbonitrile,
ethyl 2-amino-3-cyano-4-[5-(hydroxymethyl)furan-2-yl]-5,6,7,8-tetrahydroquinoline-6-carboxylate,
ethyl 2,6-dichloro-4-phenylquinoline-3-carboxylate,
ethyl 2,6-dibromo-4-phenylquinoline-3-carboxylate,
ethyl 2,6-dinitro-4-phenylquinoline-3-carboxylate,
ethyl 5-chloro-2-methyl-4-phenylquinoline-3-carboxylate,
ethyl 6-chloro-2-methyl-4-phenylquinoline-3-carboxylate,
ethyl 7-chloro-2-methyl-4-phenylquinoline-3-carboxylate,
ethyl 8-chloro-2-methyl-4-phenylquinoline-3-carboxylate,
ethyl 5-bromo-2-methyl-4-phenylquinoline-3-carboxylate,
ethyl 6-bromo-2-methyl-4-phenylquinoline-3-carboxylate,
ethyl 7-bromo-2-methyl-4-phenylquinoline-3-carboxylate,
ethyl 8-bromo-2-methyl-4-phenylquinoline-3-carboxylate,
ethyl 5-nitro-2-methyl-4-phenylquinoline-3-carboxylate,
ethyl 6-nitro-2-methyl-4-phenylquinoline-3-carboxylate,
ethyl 7-nitro-2-methyl-4-phenylquinoline-3-carboxylate,
ethyl 8-nitro-2-methyl-4-phenylquinoline-3-carboxylate,
ethyl 5-chloro-4-(morpholin-4-yl)quinoline-3-carboxylate,
ethyl 6-chloro-4-(morpholin-4-yl)quinoline-3-carboxylate,
ethyl 7-chloro-4-(morpholin-4-yl)quinoline-3-carboxylate,
ethyl 8-chloro-4-(morpholin-4-yl)quinoline-3-carboxylate,
ethyl 5-bromo-4-(morpholin-4-yl)quinoline-3-carboxylate,
ethyl 6-bromo-4-(morpholin-4-yl)quinoline-3-carboxylate,
ethyl 7-bromo-4-(morpholin-4-yl)quinoline-3-carboxylate,
ethyl 8-bromo-4-(morpholin-4-yl)quinoline-3-carboxylate,
ethyl 5-nitro-4-(morpholin-4-yl)quinoline-3-carboxylate,
ethyl 6-nitro-4-(morpholin-4-yl)quinoline-3-carboxylate,
ethyl 7-nitro-4-(morpholin-4-yl)quinoline-3-carboxylate,
ethyl 8-nitro-4-(morpholin-4-yl)quinoline-3-carboxylate,
3-(butane-1-sulfonyl)-6-chloro-4-phenylquinolin-2-ol,
2-amino-4-(5-cyano-1,2-dimethyl-1H-pyrrol-3-yl)-6-cyclopropanecarbonyl-5,6,7,8-tetrahydro-1,6-naphthyridine-3-carbonitrile,
2-amino-4-(5-chlorothiophen-2-yl)-6-cyclopropanecarbonyl-5,6,7,8-tetrahydro-1,6-naphthyridine-3-carbonitrile,
methyl 3-(2-amino-6-butanoyl-3-cyano-5,6,7,8-tetrahydro-1,6-naphthyridin-4-yl)benzoate,
ethyl 2-amino-3-cyano-4-(5-cyano-1-methyl-1H-pyrrol-3-yl)-5,6,7,8-tetrahydroquinoline-6-carboxylate,
ethyl 2-amino-3-cyano-4-(4-hydroxy-2-methoxyphenyl)-5,6,7,8-tetrahydroquinoline-6-carboxylate,
3-[5-chloro-3-(ethoxycarbonyl)-2-methylquinolin-4-yl]propanoic acid,
3-[6-chloro-3-(ethoxycarbonyl)-2-methylquinolin-4-yl]propanoic acid,
3-[7-chloro-3-(ethoxycarbonyl)-2-methylquinolin-4-yl]propanoic acid,
3-[8-chloro-3-(ethoxycarbonyl)-2-methylquinolin-4-yl]propanoic acid,
3-[5-bromo-3-(ethoxycarbonyl)-2-methylquinolin-4-yl]propanoic acid,
3-[6-bromo-3-(ethoxycarbonyl)-2-methylquinolin-4-yl]propanoic acid,
3-[7-bromo-3-(ethoxycarbonyl)-2-methylquinolin-4-yl]propanoic acid,
3-[8-bromo-3-(ethoxycarbonyl)-2-methylquinolin-4-yl]propanoic acid,
3-[5-nitro-3-(ethoxycarbonyl)-2-methylquinolin-4-yl]propanoic acid,
3-[6-nitro-3-(ethoxycarbonyl)-2-methylquinolin-4-yl]propanoic acid,
3-[7-nitro-3-(ethoxycarbonyl)-2-methylquinolin-4-yl]propanoic acid,
3-[8-nitro-3-(ethoxycarbonyl)-2-methylquinolin-4-yl]propanoic acid,
ethyl 5-chloro-4-(3-ethoxy-3-oxopropyl)-2-methylquinoline-3-carboxylate,
ethyl 6-chloro-4-(3-ethoxy-3-oxopropyl)-2-methylquinoline-3-carboxylate,
ethyl 7-chloro-4-(3-ethoxy-3-oxopropyl)-2-methylquinoline-3-carboxylate,
ethyl 8-chloro-4-(3-ethoxy-3-oxopropyl)-2-methylquinoline-3-carboxylate,
ethyl 5-bromo-4-(3-ethoxy-3-oxopropyl)-2-methylquinoline-3-carboxylate,
ethyl 6-bromo-4-(3-ethoxy-3-oxopropyl)-2-methylquinoline-3-carboxylate,
ethyl 7-bromo-4-(3-ethoxy-3-oxopropyl)-2-methylquinoline-3-carboxylate,
ethyl 8-bromo-4-(3-ethoxy-3-oxopropyl)-2-methylquinoline-3-carboxylate,
ethyl 4-(3-ethoxy-3-oxopropyl)-2-methyl-5-nitroquinoline-3-carboxylate,
ethyl 4-(3-ethoxy-3-oxopropyl)-2-methyl-6-nitroquinoline-3-carboxylate
ethyl 4-(3-ethoxy-3-oxopropyl)-2-methyl-7-nitroquinoline-3-carboxylate,
ethyl 4-(3-ethoxy-3-oxopropyl)-2-methyl-8-nitroquinoline-3-carboxylate,
ethyl 5-chloro-4-phenyl-2-propylquinoline-3-carboxylate,
ethyl 6-chloro-4-phenyl-2-propylquinoline-3-carboxylate,
ethyl 7-chloro-4-phenyl-2-propylquinoline-3-carboxylate,
ethyl 8-chloro-4-phenyl-2-propylquinoline-3-carboxylate,
ethyl 5-bromo-4-phenyl-2-propylquinoline-3-carboxylate,
ethyl 6-bromo-4-phenyl-2-propylquinoline-3-carboxylate,
ethyl 7-bromo-4-phenyl-2-propylquinoline-3-carboxylate,
ethyl 8-bromo-4-phenyl-2-propylquinoline-3-carboxylate,
ethyl 5-nitro-4-phenyl-2-propylquinoline-3-carboxylate,
ethyl 6-nitro-4-phenyl-2-propylquinoline-3-carboxylate,
ethyl 7-nitro-4-phenyl-2-propylquinoline-3-carboxylate,
ethyl 8-nitro-4-phenyl-2-propylquinoline-3-carboxylate,
3-[5-chloro-3-(ethoxycarbonyl)-2-propylquinolin-4-yl]propanoic acid,
3-[6-chloro-3-(ethoxycarbonyl)-2-propylquinolin-4-yl]propanoic acid,
3-[7-chloro-3-(ethoxycarbonyl)-2-propylquinolin-4-yl]propanoic acid,
3-[8-chloro-3-(ethoxycarbonyl)-2-propylquinolin-4-yl]propanoic acid,
3-[5-bromo-3-(ethoxycarbonyl)-2-propylquinolin-4-yl]propanoic acid,
3-[6-bromo-3-(ethoxycarbonyl)-2-propylquinolin-4-yl]propanoic acid,
3-[7-bromo-3-(ethoxycarbonyl)-2-propylquinolin-4-yl]propanoic acid,
3-[8-bromo-3-(ethoxycarbonyl)-2-propylquinolin-4-yl]propanoic acid,
3-[5-nitro-3-(ethoxycarbonyl)-2-propylquinolin-4-yl]propanoic acid,
3-[6-nitro-3-(ethoxycarbonyl)-2-propylquinolin-4-yl]propanoic acid,
3-[7-nitro-3-(ethoxycarbonyl)-2-propylquinolin-4-yl]propanoic acid,
3-[8-nitro-3-(ethoxycarbonyl)-2-propylquinolin-4-yl]propanoic acid,
ethyl 5-chloro-4-(3-ethoxy-3-oxopropyl)-2-propylquinoline-3-carboxylate,
ethyl 6-chloro-4-(3-ethoxy-3-oxopropyl)-2-propylquinoline-3-carboxylate,
ethyl 7-chloro-4-(3-ethoxy-3-oxopropyl)-2-propylquinoline-3-carboxylate,
ethyl 8-chloro-4-(3-ethoxy-3-oxopropyl)-2-propylquinoline-3-carboxylate,
ethyl 5-bromo-4-(3-ethoxy-3-oxopropyl)-2-propylquinoline-3-carboxylate,
ethyl 6-bromo-4-(3-ethoxy-3-oxopropyl)-2-propylquinoline-3-carboxylate,
ethyl 7-bromo-4-(3-ethoxy-3-oxopropyl)-2-propylquinoline-3-carboxylate,
ethyl 8-bromo-4-(3-ethoxy-3-oxopropyl)-2-propylquinoline-3-carboxylate,
ethyl 4-(3-ethoxy-3-oxopropyl)-5-nitro-2-propylquinoline-3-carboxylate,
ethyl 4-(3-ethoxy-3-oxopropyl)-6-nitro-2-propylquinoline-3-carboxylate,
ethyl 4-(3-ethoxy-3-oxopropyl)-7-nitro-2-propylquinoline-3-carboxylate,
ethyl 4-(3-ethoxy-3-oxopropyl)-8-nitro-2-propylquinoline-3-carboxylate,
3-[2-tert-butyl-5-chloro-3-(ethoxycarbonyl)quinolin-4-yl]propanoic acid,
3-[2-tert-butyl-6-chloro-3-(ethoxycarbonyl)quinolin-4-yl]propanoic acid,
3-[2-tert-butyl-7-chloro-3-(ethoxycarbonyl)quinolin-4-yl]propanoic acid,
3-[2-tert-butyl-8-chloro-3-(ethoxycarbonyl)quinolin-4-yl]propanoic acid,
3-[2-tert-butyl-5-bromo-3-(ethoxycarbonyl)quinolin-4-yl]propanoic acid,
3-[2-tert-butyl-6-bromo-3-(ethoxycarbonyl)quinolin-4-yl]propanoic acid,
3-[2-tert-butyl-7-bromo-3-(ethoxycarbonyl)quinolin-4-yl]propanoic acid,
3-[2-tert-butyl-8-bromo-3-(ethoxycarbonyl)quinolin-4-yl]propanoic acid,
3-[2-tert-butyl-5-nitro-3-(ethoxycarbonyl)quinolin-4-yl]propanoic acid,
3-[2-tert-butyl-6-nitro-3-(ethoxycarbonyl)quinolin-4-yl]propanoic acid,
3-[2-tert-butyl-7-nitro-3-(ethoxycarbonyl)quinolin-4-yl]propanoic acid,
3-[2-tert-butyl-8-nitro-3-(ethoxycarbonyl)quinolin-4-yl]propanoic acid,
ethyl 2-(tert-butyl)-5-chloro-4-(3-ethoxy-3-oxopropyl)quinoline-3-carboxylate,
ethyl 2-(tert-butyl)-6-chloro-4-(3-ethoxy-3-oxopropyl)quinoline-3-carboxylate,
ethyl 2-(tert-butyl)-7-chloro-4-(3-ethoxy-3-oxopropyl)quinoline-3-carboxylate
ethyl 2-(tert-butyl)-8-chloro-4-(3-ethoxy-3-oxopropyl)quinoline-3-carboxylate,
ethyl 5-bromo-2-(tert-butyl)-4-(3-ethoxy-3-oxopropyl)quinoline-3-carboxylate,
ethyl 6-bromo-2-(tert-butyl)-4-(3-ethoxy-3-oxopropyl)quinoline-3-carboxylate,
ethyl 7-bromo-2-(tert-butyl)-4-(3-ethoxy-3-oxopropyl)quinoline-3-carboxylate,
ethyl 8-bromo-2-(tert-butyl)-4-(3-ethoxy-3-oxopropyl)quinoline-3-carboxylate,
ethyl 2-(tert-butyl)-4-(3-ethoxy-3-oxopropyl)-5-nitroquinoline-3-carboxylate,
ethyl 2-(tert-butyl)-4-(3-ethoxy-3-oxopropyl)-6-nitroquinoline-3-carboxylate,
ethyl 2-(tert-butyl)-4-(3-ethoxy-3-oxopropyl)-7-nitroquinoline-3-carboxylate, and
ethyl 2-(tert-butyl)-4-(3-ethoxy-3-oxopropyl)-8-nitroquinoline-3-carboxylate.

4. The method of claim 1, wherein the mammalian cell is an immune cell, optionally wherein the immune cell is an innate or adaptive immune cell selected from the group consisting of hematopoietic progenitor cell, B lymphocyte, T lymphocyte, natural killer cell, and myeloid lineage cell.

5. (canceled)

6. The method of claim 1, wherein at least one of the following applies:
i. the mammalian cell is a keratinocyte,
ii. the at least one compound is an Akt agonist with selectivity for a single isoform of Akt kinase selected from the group consisting of Akt1, Akt2, and Akt3;
iii. the at least one compound enhances Akt phosphatase resistance.

7. (canceled)

8. (canceled)

9. The method of claim 1, wherein at least one of the following applies:
i. the mammalian cell is contacted ex vivo with the at least one compound;
ii. the contacted mammalian cell is reintroduced in vivo;
iii. the mammalian cell is present in vivo.

10. (canceled)

11. (canceled)

12. (canceled)

13. The method of claim 1, wherein:
i. the cardiovascular condition comprises myocardial infarction or stroke,
ii. the pulmonary condition comprises acute lung injury or ventilator-induced lung injury; or
iii. the metabolic disease comprises an acute metabolic disease.

14. (canceled)

15. (canceled)

16. A method of:
i. treating cancer in a subject;
ii. treating or healing a skin condition or a wound in a subject; or
iii. promoting tissue replacement, promoting tissue regeneration, or treating degenerative diseases in a subject,
the method comprising:
(a) administering to the subject a therapeutically effective amount of the at least one compound of formula (Ia), (Ib), (II), (III), and (IV), or a salt, solvate, prodrug, enantiomer, diastereoisomer or tautomer thereof; or
(b) administering to the subject a therapeutically effective amount of cells pre-treated with the at least one compound of formula (Ia), (Ib), (II), (III), and (IV), or a salt, solvate, prodrug, enantiomer, diastereoisomer or tautomer thereof: 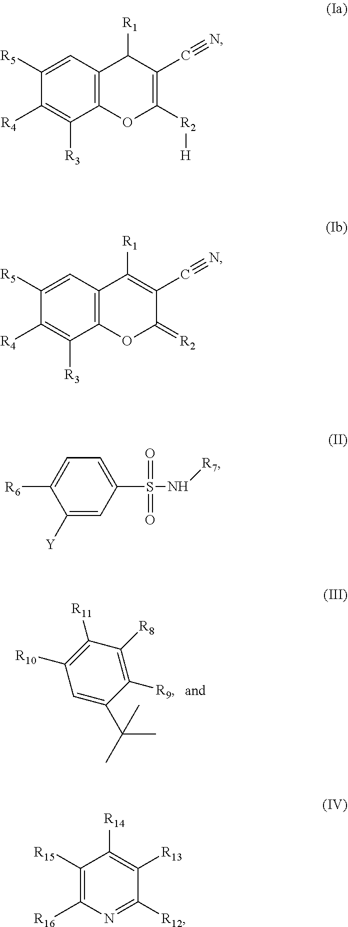
wherein:
each occurrence of R₁ is independently selected from the group consisting of —H and —CH(CN)₂;
R₂ is NH;
each occurrence of R₃ is independently selected from the group consisting of hydrogen, hydroxyl, halogen, nitro, optionally substituted C₁-C₆ alkyl, haloalkyl, optionally substituted C₁-C₆ alkoxy, optionally substituted C₃-C₈ cycloalkyl, optionally substituted C₂-C₆ alkenyl, optionally substituted C₂-C₆ alkynyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₄ is independently selected from the group consisting of hydrogen, hydroxyl, halogen, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
each occurrence of R₅ is independently selected from the group consisting of hydrogen, hydroxyl, halogen, nitro, optionally substituted C₁-C₆ alkyl, optionally substituted C₃-C₈ cycloalkyl, optionally substituted C₂-C₈ alkenyl, optionally substituted C₂-C₈ alkynyl, and optionally substituted C₁-C₆ alkoxy,
wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₆ is selected from the group consisting of hydrogen, halogen, optionally substituted C₁-C₆ alkyl, haloalkyl, optionally substituted C₃-C₈ cycloalkyl, optionally substituted phenyl, optionally substituted heteroaryl, and —NR′C(═O)R′, wherein R′ is selected from the group consisting of H, optionally substituted C₁-C₆ alkyl, optionally substituted phenyl, and optionally substituted C₃-C₆ heterocyclyl,
wherein each optional substituent in R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
Y is hydrogen, or Y and R₆ taken together with the atoms to which they are bound form an optionally substituted 4-7 membered heterocyclyl,
wherein each optional substituent in Y and R₆ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₇ is selected from the group consisting of optionally substituted C₅-C₇ cycloalkyl, optionally substituted aryl, optionally substituted heteroaryl, and optionally substituted C₅-C₇ heterocyclyl,
wherein the heteroaryl is selected from the group consisting of thiadiazolyl, thiazolyl, oxazolyl, diazolyl, imidazolyl, triazinyl, thiazolyl, isothiazolyl, and pyridinyl, and
wherein each optional substituent in R₇ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₈ is selected from the group consisting of hydrogen, optionally substituted C₁-C₆ alkyl, and —C(═O)OR′, wherein R′ is selected from the group consisting of H, C₁-C₆ alkyl, C₃-C₈ cycloalkyl, C₂-C₈ alkenyl, and C₂-C₈ alkynyl,
wherein each optional substituent in R₈ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₉ is selected from the group consisting of hydrogen, hydroxyl, and C₁-C₆ alkoxy;
R₁₀ is selected from the group consisting of: 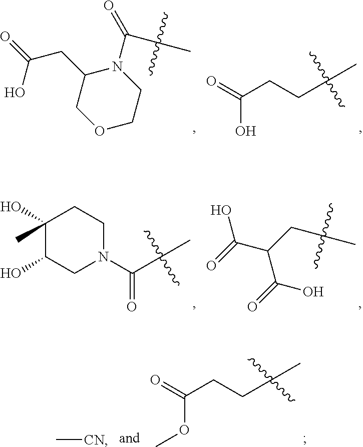
R₁₁ is selected from the group consisting of hydrogen and —NR′(C═O)R′, wherein each R′ is independently selected from the group consisting of H, C₁-C₆ alkyl, C₃-C₈ cycloalkyl, C₂-C₈ alkenyl, and C₂-C₈ alkynyl;
R₁₂ is selected from the group consisting of hydrogen, hydroxyl, —NH₂, optionally substituted C₁-C₆ alkyl, optionally substituted C₃-C₈ cycloalkyl, optionally substituted C₂-C₆ alkenyl, optionally substituted C₂-C₆ alkynyl, optionally substituted C₁-C₆ alkoxy, halogen, and haloalkyl,
wherein each optional substituent in R₁₂ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₃ is selected from the group consisting of —CN, —SO₂R′, and —C(═O)OR′, wherein R′ is selected from the group consisting of H, C₁-C₆ alkyl, C₃-C₈ cycloalkyl, C₂-C₈ alkenyl, and C₂-C₈ alkynyl;
R₁₄ is selected from the group consisting of —(CH₂)ₙC(═O)OR′, optionally substituted C₅-C₇ cycloalkyl, optionally substituted aryl, optionally substituted morpholinyl, and optionally substituted heteroaryl, wherein R′ is selected from the group consisting of H and C₁-C₆ alkyl,
wherein n is an integer from 1-6,
wherein the heteroaryl is selected from the group consisting of furanyl, pyrrolyl, and thiofuranyl, and
wherein each optional substituent in R₁₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₁₅ and R₁₆ taken together with the atoms to which they are bound form an optionally substituted 4-8 membered heterocyclyl, optionally substituted aryl, or optionally substituted heteroaryl,
wherein each optional substituent in R₁₅ and R₁₆ is independently selected from the group consisting of halogen, C₁-C₆ alkyl, C₃-C₈ cycloalkyl, C₂-C₈ alkenyl, C₂-C₈ alkynyl, phenyl, hydroxyl, nitro, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

17. (canceled)

18. (canceled)

19. The method of claim 16, wherein the administration duration is equal to or less than 14 days.

20. The method of claim 16, wherein the administration does not cause any significant deleterious or unwanted cell multiplication in the subject.

21. The method of claim 16, which does not cause significant systemic exposure of the at least one compound in the subject.

22. The method of claim 16, wherein the treatment enhances formation of new connective tissue and/or microscopic blood vessels in the subject.

23. The method of claim 16, wherein the subject is a mammal, optionally wherein the mammal is a human.

24. (canceled)

25. (canceled)

26. (canceled)

27. A compound of formula (Va) or (Vb), or a salt, solvate, prodrug, enantiomer, diastereoisomer or tautomer thereof: 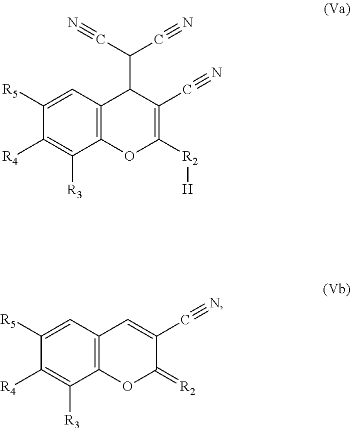
wherein:
R₂ is NH;
R₃ is selected from the group consisting of hydrogen, optionally substituted C₁-C₆ alkyl, optionally substituted C₃-C₈ cycloalkyl, optionally substituted C₂-C₆ alkenyl, optionally substituted C₂-C₆ alkynyl, hydroxyl, optionally substituted C₁-C₆ alkoxy, halogen, haloalkyl, nitro, and —C(═O)OR′, wherein R′ is selected from the group consisting of H, optionally substituted C₁-C₆ alkyl, optionally substituted C₃-C₈ cycloalkyl, optionally substituted C₂-C₈ alkenyl, and optionally substituted C₂-C₈ alkynyl,
wherein each optional substituent in R₃ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₄ is selected from the group consisting of hydrogen, halogen, optionally substituted C₁-C₆ alkyl, optionally substituted C₃-C₈ alkyl, optionally substituted C₂-C₈ alkenyl, optionally substituted C₂-C₈ alkynyl, optionally substituted C₁-C₆ alkoxy, and hydroxyl,
wherein each optional substituent in R₄ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl;
R₅ is selected from the group consisting of hydrogen, halogen, optionally substituted C₁-C₆ alkyl, optionally substituted C₃-C₃ cycloalkyl, optionally substituted C₂-C₈ alkenyl, optionally substituted C₂-C₈ alkynyl, nitro, hydroxyl, and optionally substituted C₁-C₆ alkoxy,
wherein each optional substituent in R₅ is independently selected from the group consisting of halogen, C₁-C₆ alkyl, C₃-C₈ cycloalkyl, C₂-C₈ alkenyl, C₂-C₈ alkynyl, phenyl, hydroxyl, C₁-C₆ alkoxy, and —C(═O)OR″, wherein R″ is selected from the group consisting of H, C₁-C₆ alkyl, C₃-C₈ cycloalkyl, C₂-C₈ alkenyl, and C₂-C₈ alkynyl.

28. A pharmaceutical composition comprising at least one pharmaceutically acceptable carrier and at least one compound of claim 27.
